(19) United States
(12) Patent Application Publication
(10) Pub. No.: US 2004/0082664 A1
(43) Pub. Date: Apr. 29, 2004

(54) Derivatives of hydroxyphenyl, a method for preparing thereof and their pharmaceutical composition

(76) Inventors: Jongwha Won, Seongnam-si (KR); Keunhyeung Lee, Seoul (KR); Seehyoung Park, Suwon-si (KR); Sung-Joo Kim, Yongin-si (KR); Su-Young Yun, Seongnam-si (KR); Mi-Ae Kang, Gunpo-si (KR); Yun-Gyoung Hur, Hwaseong-gun (KR); Jeehee Youn, Seoul (KR); Yungdae Yun, Seoul (KR); Doohong Park, Seoul (KR); Jaetaek Oh, Seoul (KR)

Correspondence Address: MUSERLIAN AND LUCAS AND MERCANTI, LLP, 475 PARK AVENUE SOUTH, NEW YORK, NY, 10016, US

(21) Appl. No.: 10/411,772

(22) Filed: Apr. 11, 2003

(30) Foreign Application Priority Data
Apr. 15, 2002 (KR) KR/2002-0020481

(51) Int. Cl. A61K 31/137

(52) U.S. Cl. 514/649

(57) ABSTRACT

The present invention relates to derivatives of hydroxyphenyl, a method for preparing thereof and their pharmaceutical composition, more particularly the compounds of the present invention specifically inhibit the activation of T lymphocyte by src homology region 2(SH2) domain of T lymphocyte (lck), so that they can be used for the treatment, prevention and/or diagnosis of graft rejection, autoimmune diseases, inflammatory diseases, etc.

### TECHNICAL FIELD

The present invention relates to derivatives of hydroxyphenyl represented by following formula 1, a method for preparing thereof and pharmaceutical composition.

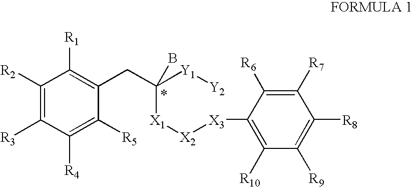

Wherein, R₁, R₂, R₃, R₄, R₅, R₆, R₇, R₈, R₉, R₁₀, X₁, X₂, X₃, Y₁, Y₂, B and * are same as defined in the description.

### BACKGROUND OF THE INVENTION

Immunosuppressive drugs are widely used in treating transplant rejection and autoimmune diseases. During immune responses, the number of leukocytes including T-lymphocytes, B-lymphocytes, monocytes and polymorphonuclear cells rapidly increases. Most classical immunosuppressive drugs are developed aiming at the above, and thus, suppress the immune response involving lymphocyte activation and proliferation by inhibiting cytokine expression and cell metabolism. In general, the classical immunosuppressive drugs can be divided into metabolic inhibitors blocking purine/pyrimidine synthesis and steroid inhibitors suppressing the expression of cytokine genes (YOON, Young-sik, Journal of Korean Kidney Society, Vol 13, appendix No. 8:S66-S85, 1994; N. Perico and G. Remuzzi. Drugs 54(4):533-570, 1997). Immunosuppressants suppressing DNA and RNA synthesis can be exemplified by azathioprine, mycophenolic acid, brequinar, deoxyspergualin, etc, and steroid inhibitors can be exemplified by corticosteroid, prednisone, etc. However, since these immunosuppressants do not have specificity towards leukocytes and basically act on most of actively proliferating cells including hematopoietic cells, they accompany various side effects such as functional disorder in the heart, liver and kidney, hematopoiesis. Immunosuppressants such as cyclosporin A, FK506 and rapamycin were developed after 1980 and inhibit T cell activation and proliferation by blocking the T lymphocyte antigen receptor (TCR)-induced, and IL-2 receptor-mediated signal transduction, respectively. Cyclosporin A and FK506 suppress the function of calcineurin, so that they prevent the translocation of the transcription activating factor NF-AT from cytosol to nucleus, resulting in the failure of IL-2 expression (C. T. Walsh et al. J. Biol. Chem., 267:13115. 1992; S. L. Schreiber and G. R. Crabtree. Immunol. Today. 13:136, 1992). Rapamycin does not suppress TCR-induced IL-2 expression, but suppresses T lymphocytes from entering G1 to S phase by binding with and inhibiting the function of mTor, a major signal transducer in IL-2 receptor-mediated signaling. Cyclosporin A, FK506 and rapamycin have much less side effects than those of classical immunosuppressive drugs in the view that they target the signal transduction in T-lymphocyte. However, they still cause some problems in the heart, kidney, liver and stomach and this is ascribed from the ubiquitous distribution of the target molecules of these drugs.

Nowadays, combinations of cyclosporine A, azathioprine, prednisone or corticosteroids such as methylprednison and cyclophosphamide are being used in controlling allograft rejection. Among them, cyclosporine A is the most potent and commonly used immunosuppressant, which has brought an innovation in the sphere of transplant surgery. Other recently launched drugs including FK506, rapamycin, mycophenolic acid, 15-deoxyspergualin, mizoribine, misoprostol, OKT3, and an antibody against the interleukin-2 (hereinafter abbreviated into “IL-2”) receptor have also been used in controlling or preventing transplant rejection (Briggs, Immunology letters July 29(1-2): 89-94, 1991; FASEB 3:3411, 1989).

In addition to the above-mentioned generally used immunosuppressants, therapeutics for arthritis, an antoimmune disease, include non-steroidal anti-inflammatroy drugs (NSAID) and disease modifying anti-rhuematic drugs (DMARD) (J. P. Case, Am. J. Ther., 8:123-143; 163-179, 2001). NSAIDs are effective in relieving symptoms of arthritis and its progression by inhibiting cyclooxygenase (COX), which plays an important role in inflammatory reactions. However, since NSAIDs do not prevent the fundamental cause of arthritis, they must be used with DMARDs. DMARDs are exemplified by cell metabolic inhibitor, steroids, TNF-α signaling inhibitors. TNF-α-mediated inflammation can be blocked by TNF-α signaling inhibitor, leflunomide, or by interruption of TNF-α/TNF-α receptor interactions by anti TNF-α antibody or soluble TNF-α receptor. Therapeutics currently being used for rheumatoid arthritis include steroids, NSAIDs such as ibuprofen, diclofenac, ketoprofen and naproxen, in particular cyclooxygenase II-specific NSAIDs such as celecoxib and rofecoxib, T-cell signal transduction inhibitors such as cyclosporine, metabolic inhibitors such as methotrexate, leflunomide, azathioprine and cyclophosphamide, and TNF-α targeting protein/antibody such as etanercept and infliximab.

As disclosed above, the target cells on which immunosuppressive drugs should act are leukocytes and the degree of drug-induced side effects might be dependent on spectrum of cells on which drugs can act. T lymphocytes play the pivotal role in immune responses and, therefore, side effects can be minimized if drugs are developed towards T lymphocytes only. Lymphocyte-specific cytoplasmic protein tyrosine kinase. (hereinafter abbreviated into “lck”), a Src family protein tyrosine kinase, is restricted to T cells and NK cells, and plays an essential role in TCR-induced T cell activation, growth and differentiation (Xu and Littman, Cell 74: 633-643, 1993). In order to accomplish the above stated essential role of lck, protein-to-protein interactions through the SH2- and SH3-domains as well as lck kinase activity are important. This was proved by observation, wherein lck with modified SH₂ domain so that it does not recognize phosphotyrosine lost its ability to activate T cells. Inhibition of Lck SH2-mediated protein interaction prevents TCR-induced phosphorylation of ξ chain and ZAP70, cytoplasmic Ca⁺⁺ mobilization, and IL-2 expression (Straus et al., J. Biol. Chem., 271: 9976-9981, 1996; Lewis et al., J. Immunol., 159: 2292-2300, 1997). Therefore, T cell activation can be selectively suppressed by blocking Lck SH2-mediated protein-to-protein interactions. The inhibitors of Lck SH2-mediated protein-to-protein interaction can be applied for the treatment of various diseases caused by the uncontrolled overreaction of T lymphocytes.

### DISCLOSURE OF THE INVENTION

It is an object of the present invention to provide compounds represented by formula 1, their pharmaceutically acceptable salts and preparation method thereof.

It is another object of the present invention to provide a pharmaceutical composition for use in inhibiting activity of SH2 domain in T lymphocyte cell kinase, Lck, comprising the compounds or pharmaceutically acceptable salts of formula 1 as an effective ingredient.

It is another object of the present invention to provide a pharmaceutical composition for use in inhibiting immune response, comprising the compounds or pharmaceutically acceptable salts of formula 1 as an effective ingredient.

It is another object of the present invention to provide a pharmaceutical composition for use in inflammation, comprising the compounds or pharmaceutically acceptable salts of formula 1 as an effective ingredient.

It is another object of the present invention to provide a pharmaceutical composition for use in treating arthritis, comprising the compounds or pharmaceutically acceptable salts of formula 1 as an effective ingredient.

In order to accomplish the aforementioned objects, the present invention provides compounds represented by following formula 1 and their pharmaceutically acceptable salts.

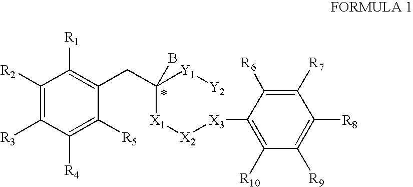

Wherein, R₁, R₂ , R₃ , R₄ and R₅ are all independent of each other and at least one of them is hydroxyl group, others are selected from the group consisting of hydrogen; halogen atom; C₁˜C₃ alkoxy; aldehyde; carboxyl; amino; trifluoromethyl; and nitro;

R₆, R₇, R₈, R₉ and R₁₀, are also independent of each other and at least one of them is hydroxy group, others are selected from the group consisting of hydrogen; halogen atom; C₁˜C₃ alkoxy; aldehyde; carboxyl; amino; trifluoromethyl; and nitro;

X₁ is O; S; —NH; —N(CH₃)—; —N(CH₂CH₃)—; or —NHNH—;

X₂ is —CH₂—; —C(═O)—; —C(═S)—; or —C(═O)—NH—;

X₃ is selected from the group consisting of

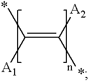

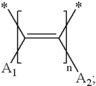

and —(CH₂)ₘ—;

wherein A₁ is hydrogen; C₁˜C₄ straight or branched alkyl; thiol; phenyl; cyano; or C₁˜C₃ alkoxycarbonyl,

A₂ is hydrogen; or C₁˜C₄ straight or branched alkyl, n is 0, 1 or 2, m is 0, 1 or 2;

Y₁ is selected from the group consisting of hydrogen; —CH₂—; —C(═O)—; —C(═S)—; C₁˜C₄ straight or branched alkyl or amine substituted with aryl;

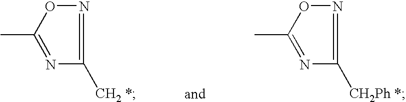

Y₂ does not exist or is —NZ₁₁Z₁₂; —O—Z₂; or —S—Z₂;

wherein Z₁₁ and Z₁₂ are independent each other and can be hydrogen; amine optionally substituted with t-butoxycarbonyl; C₁˜C₁₂ straight or branched alkyl; aryl; cycloalkyl; or heteroalkyl;

Z₂ is hydrogen; C₁˜C₁₂ straight or branched alkyl; aryl; cycloalkyl; or heteroalkyl;

B is hydrogen or alkyl;

* represents a chiral carbon.

Also, the compounds of formula 1 represent both R-form and S-form stereoisomer, and comprise both stereoisomer compounds and racemic mixture.

Preferably, R₂, R₃, R₈ and R₉ are hydroxy group.

More preferably, R₁, R₄ and R₅ are hydrogen; R₂ and R₃ are hydroxy; R₆, R₇ and R₁₀ are hydrogen; R₈ and R₉ are hydroxy; X₁ is O, S, —NH— or —N(CH₃)—; X₂ is —CH₂—, —C(═O)— or —C(═S)—; X₃ is —CH═CH—; Y₁ may or may not be 0; Y₂ is C₁˜C₄ alkoxy; —NH₂ or hydroxy; B is 0.

Preferably, the compounds of formula 1 comprise:

1) 3-(3,4-dihydroxy-phenyl)-(R)-2-[3-trans-3,4-dihydroxy-phenyl)-acryloylamino]-propionic acid methyl ester;

2) 3-(3,4-dihydroxy-phenyl)-(R)-2-[3-trans-3,4-dihydroxy-phenyl)-acryloylamino]-propionic acid;

3) 3-(3,4-dihydroxy-phenyl)-(R)-2-[3-trans-3,4-dihydroxy-phenyl)-acryloylamino]-propionic acid ethyl ester;

4) 3-(3,4-dihydroxy-phenyl)-(R)-2-[3-trans-(3,4-dihydroxy-phenyl)-acryloylamino]-propionic acid propyl ester;

5) 3-(3,4-dihydroxy-phenyl)-(R)-2-[3-trans-(3,4-dihydroxy-phenyl)-acryloylamino]-propionic acid isopropyl ester;

6) 3-(3,4-dihydroxy-phenyl)-(R)-2-[3-trans-(3,4-dihydroxy-phenyl)-acryloylamino]-propionic acid tert-butyl ester;

7) 3-(3,4-dihydroxy-phenyl)-(R)-2-[3-trans-(3,4-dihydroxy-phenyl)-acryloylamino]-propioneamide;

8) 3-(3,4-dihydroxy-phenyl)-(S)-2-[3-trans-(3,4-dihydroxy-phenyl)-acryloylamino]-propionic acid methyl ester;

9) 3-(3,4-dihydroxy-phenyl)-(S)-2-[3-trans-(3,4-dihydroxy-phenyl)-acryloylamino]-propionic acid;

10) 3-(3,4-dihydroxy-phenyl)-(S)-2-[3-trans-(3,4-dihydroxy-phenyl)-acryloylamino]-propionic acid ethyl ester;

11) 3-(3,4-dihydroxy-phenyl)-(S)-2-[3-trans-(3,4-dihydroxy-phenyl)-acryloylamino]-propionic acid propyl ester;

12) 3-(3,4-dihydroxy-phenyl)-(S)-2-[3-trans-(3,4-dihydroxy-phenyl)-acryloylamino]-propionic acid isopropyl ester;

13) 3-(3,4-dihydroxy-phenyl)-(S)-2-[3-trans-(3,4-dihydroxy-phenyl)-acryloylamino]-propionic acid tert-butyl ester;

14) 3-(3,4-dihydroxy-phenyl)-(S)-2-[3-trans-(3,4-dihydroxy-phenyl)-acryloylamino]-propioneamide;

15) 3-(3,4-dihydroxy-phenyl)-(R)-2-[3-trans-(3,4-dihydroxy-phenyl)-thioacryloylamino]-propionic acid methyl ester;

16) 3-(3,4-dihydroxy-phenyl)-(R)-2-[3-trans-(3,4-dihydroxy-phenyl)-thioacryloylamino]-propionic acid;

17) 3-(3,4-dihydroxy-phenyl)-(R)-2-[3-trans-(3,4-dihydroxy-phenyl)-thioacryloylamino]-propionic acid ethyl ester;

18) 3-(3,4-dihydroxy-phenyl)-(R)-2-[3-trans-(3,4-dihydroxy-phenyl)-thioacryloylamino]-propionic acid propyl ester;

19) 3-(3,4-dihydroxy-phenyl)-(R)-2-[3-trans-(3,4-dihydroxy-phenyl)-thioacryloylamino]-propionic acid isopropyl ester;

20) 3-(3,4-dihydroxy-phenyl)-(R)-2-[3-trans-(3,4-dihydroxy-phenyl)-thioacryloylamino]-propionic acid tertbutyl ester;

21) 3-(3,4-dihydroxy-phenyl)-(R)-2-[3-trans-(3,4-dihydroxy-phenyl)-thioacryloylamino]-propioneamide;

22) 3-(3,4-dihydroxy-phenyl)-(S)-2-[3-trans-(3,4-dihydroxy-phenyl)-thioacryloylamino]-propionic acid methyl ester;

23) 3-(3,4-dihydroxy-phenyl)-(S)-2-[3-trans-(3,4-dihydroxy-phenyl)-thioacryloylamino]-propionic acid;

24) 3-(3,4-dihydroxy-phenyl)-(S)-2-[3-trans-(3,4-dihydroxy-phenyl)-thioacryloylamino]-propionic acid ethyl ester;

25) 3-(3,4-dihydroxy-phenyl)-(S)-2-[3-trans-(3,4-dihydroxy-phenyl)-thioacryloylamino]-propionic acid propyl ester;

26) 3-(3,4-dihydroxy-phenyl)-(S)-2-[3-trans-(3,4-dihydroxy-phenyl)-thioacryloylamino]-propionic acid isopropyl ester;

27) 3-(3,4-dihydroxy-phenyl)-(S)-2-[3-trans-(3,4-dihydroxy-phenyl)-thioacryloylamino]-propionic acid tertbutyl ester;

28) 3-(3,4-dihydroxy-phenyl)-(S)-2-[3-trans-(3,4-dihydroxy-phenyl)-thioacryloylamino]-propioneamide;

29) 3-(3,4-dihydroxy-phenyl)-(R)-2-{[3-trans-(3,4-dihydroxy-phenyl)-acryloyl]-methyl-amino}-propionic acid methyl ester;

30) 3-(3,4-dihydroxy-phenyl)-(R)-2-{[3-trans-(3,4-dihydroxy-phenyl)-acryloyl]-methyl-amino}-propionic acid;

31) 3-(3,4-dihydroxy-phenyl)-(R)-2-{[3-trans-(3,4-dihydroxy-phenyl)-acryloyl]-methyl-amino}-propionic acid ethyl ester;

32) 3-(3,4-dihydroxy-phenyl)-(R)-2-{[3-trans-(3,4-dihydroxy-phenyl)-acryloyl]-methyl-amino}-propionic acid propyl ester;

33) 3-(3,4-dihydroxy-phenyl)-(R)-2-{[3-trans-(3,4-dihydroxy-phenyl)-acryloyl]-methyl-amino}-propionic acid isopropyl ester;

34) 3-(3,4-dihydroxy-phenyl)-(R)-2-{[3-trans-(3,4-dihydroxy-phenyl)-acryloyl]-methyl-amino}-propionic acid tert-butyl ester;

35) 3-(3,4-dihydroxy-phenyl)-(R)-2-[3-trans-(3,4-dihydroxy-phenyl)-acryloyl]-methyl-amino]-propioneamide:

36) 3-(3,4-dihydroxy-phenyl)-(S)-2-{[3-trans-(3,4-dihydroxy-phenyl)-acryloyl]-methyl-amino}-propionic acid methyl ester;

37) 3-(3,4-dihydroxy-phenyl)-(S)-2-{[3-trans-(3,4-dihydroxy-phenyl)-acryloyl]-methyl-amino}-propionic acid;

38) 3-(3,4-dihydroxy-phenyl)-(S)-2-{[3-trans-(3,4-dihydroxy-phenyl)-acryloyl]-methyl-amino}-propionic acid ethyl ester;

39) 3-(3,4-dihydroxy-phenyl)-(S)-2-{[3-trans-(3,4-dihydroxy-phenyl)-acryloyl]-methyl-amino}-propionic acid propyl ester;

40) 3-(3,4-dihydroxy-phenyl)-(S)-2-{[3-trans-(3,4-dihydroxy-phenyl)-acryloyl]-methyl-amino}-propionic acid isopropyl ester;

41) 3-(3,4-dihydroxy-phenyl)-(S)-2-{[3-trans-(3,4-dihydroxy-phenyl)-acryloyl]-methyl-amino}-propionic acid tert-butyl ester;

42) 3-(3,4-dihydroxy-phenyl)-(S)-2-[3-trans-(3,4-dihydroxy-phenyl)-acryloyl]-methyl-amino]-propioneamide;

43) 3-(3,4-dihydroxy-phenyl)-N-[2-trans-(3,4-dihydroxy-phenyl)-ethyl]-acrylamide;

44) 3-(3,4-dihydroxy-phenyl)-N-[2-trans-(3,4-dihydroxy-phenyl)-ethyl]-N-methyl-acrylamide;

45) (R)-2-[trans-3-(3,4-dihydroxy-phenyl)-acryloylamino]-3-(4-hydroxy-phenyl)-propionic acid methyl ester;

46) (R)-2-[trans-3-(3,4-dihydroxy-phenyl)-acryloylamino]-3-(4-hydroxy-phenyl)-propionic acid;

47) (S)-2-[trans-3-(3,4-dihydroxy-phenyl)-acryloylamino]-3-(4-hydroxy-phenyl)-propionic acid methyl ester;

48) (S)-2-[trans-3-(3,4-dihydroxy-phenyl)-acryloylamino]-3-(4-hydroxy-phenyl)-propionic acid;

49) (S)-2-[3-(3,4-dihydroxy-benzyl)-ureido]-3-(3,4-dihydroxy-phenyl)-propionic acid methyl ester;

50) 3-(3,4-dihydroxy-phenyl)-2-[2-(3,4-dihydroxy-phenyl)-acetylamino]-propionic acid methyl ester;

51) 2-(3,4-dihydroxy-benzoylamino)-3-(3,4-dihydroxy-phenyl)-propionic acid methyl ester;

52) 3-(3,4-dihydroxy-phenyl)-2-[3-(3,4-dihydroxy-phenyl)-propionylamino]-propionic acid methyl ester;

53) 3-(3,4-dihydroxy-phenyl)-2-[3-(3,4-dihydroxy-phenyl)-arylamino]-propionic acid methyl ester;

54) (R)-3-(3,4-dihydroxy-phenyl)-acrylic acid 2-(3,4-dihydroxy-phenyl)-1-methoxycarbonyl ethyl ester;

55) (R)-3-(3,4-dihydroxy-phenyl)-acrylic acid 2-(3,4-dihydroxy-phenyl)-1-propoxycarbonyl ethyl ester;

56) (R)-3-(3,4-dihydroxy-phenyl)-acrylic acid 2-(3,4-dihydroxy-phenyl)-1-tert-butoxycarbonyl ethyl ester;

57) (R)-3-(3,4-dihydroxy-phenyl)-acrylic acid 2-(3,4-dihydroxy-phenyl)-1-carbamoyl ethyl ester; and

58) (R)-3-(3,4-dihydroxy-phenyl)-acrylic acid 2-(3,4-dihydroxy-phenyl)-1-isopropylcarbamoyl ethyl ester.

In a preferred embodiment, in order to examine the correlation of chemical structure of the derivatives of the present invention, which were presented as the chemical formula 1, with their activities, the present inventors investigated inhibitory effects of the derivatives of the present invention on interaction between the lck SH2 and its cognate peptide PYEEI. Judging from the results, both phenyl ring of the above derivatives must have at least one hydroxyl group, or preferably at least two hydroxyl groups, and when X₁, X₂ and X₃ make a plane, the derivatives showed more excellent activity. In addition, we confirmed that when X₁ and X₂ formed an amide, thioamide or ester, their activities were similar, and when X₃ had double bond, they strongly inhibited Lck SH2-pYEEI binding and IL-2 expression.

In addition, when Y₁ and Y₂ formed an amide group, or Y₂ was methylester, isopropylester, n-propylester, tert-butylester, or ethylester group, derivatives showed more stronger inhibitory activities in in vitro bioassays than those with carboxyl terminal group at Y₁ and Y₂. Accordingly, as shown by the derivatives of the present invention, substitution of the carboxyl group with other hydrophobic functional groups did not weaken the inhibitory activities of the derivatives on Lck SH2-pYEEI interaction, and alternately increased the activity in in vitro bioassays such as IL-2 luciferase assay presumably by increasing hydrophobicity. On the other hand, if Y₁ and Y₂ didn't have a carboxyl group, that is to be eliminated, the inhibitory activities of the derivatives were largely decreased. When X₁ and X₂ formed an amide group, stereoisomer R form was superior to S form in its inhibitory ability in various in vitro binding and bioassays. However, since other chemicals showed almost equal value, stereoisomer was confirmed not to have a large effect on IL-2 promoter assays.

In an alternatively preferred embodiment, the chemicals of the present invention inhibited or reduced the onset of arthritis in collagen II-induced mouse arthritis model. Since the chemicals of the embodiments were administered when the joints started swollen, we conclude that the chemicals have therapeutic as well as prophylactic effects on arthritis.

The compounds of formula 1 of the present invention can be used as forms of pharmaceutically acceptable salts, wherein the salts are acid addition salts formed by pharmaceutically acceptable free acid. _Whether it is inorganic or organic, a free acid can be used if it is pharmaceutically acceptable. Examples of the inorganic free acid include hydrochloric acid, bromic acid, sulfuric acid, and phosphoric acid. Available organic free acids are exemplified by citric acid, acetic acid, lactic acid, tartaric acid, maleic acid, fumaric acid, formic acid, propionic acid, oxalic acid, trifluoroacetic acid, benzoic acid, gluconic acid, methane sulfonic acid, glyconic acid, succinic acid, 4-toluenesulfonic acid, galuturonic acid, embonic acid, glutamic acid and aspartic acid.

In accordance with another aspect of the present invention, there is provided a method for preparing the compounds of formula 1.

When final product includes an amide group by bond between Y₁ and Y₂, the compounds of formula 1 which comprise intramolecular amide bond, can be prepared from condensating of amine compounds with carboxyl compound.

In accordance with a preferred embodiment, the compounds comprising amide bond of formula 1 which comprise intramolecular amide bond, prepared from condensating amine compound of formula 2 with carboxyl compound of formula 3 in the presence of coupling reagent and base, as represented in the following chemical reaction 1.

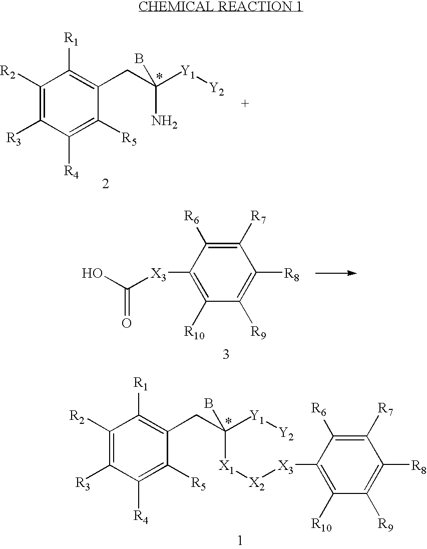

Wherein, R₁, R₂, R₃, R₄, R₅, R₆, R₇, R₈, R₉, R₁₀, X₁, X₂, X₃, Y₁, Y₂, B and * are same as defined above.

Coupling reagent is selected from the group consisting of commonly used benzotriazole-1-yl-oxytripyrollidine phosphonium hexafluorophosphate (PyBOP) and bromo-1-tripyrrolidine phosphonium hexafluorophosphate(PyBroP), but not limited thereto.

Examples of base are p-dimethylaminopyridine(DMAP), triethylamine(TEA) or diisopropylamine, etc. The base can promote the condensation.

The compounds of formula 1 can be prepared by converting various functional groups dihydroxylphenylalanine, tyrosine, dopamine, etc of the compounds of formula 2 with conventional methods.

In accordance with the other preferred embodiment, the compounds of the present invention can be prepared from converting the carboxyl compound of formula 2 by a various esterification or amidation, and then reacting the converted compound with the compound of formula 3 by the same method as represented chemical scheme 1.

In accordance with a preferred embodiment, the compounds of formula 1 comprising thio(C═S) bond were prepared from protecting carboxyl group and hydroxy group of the compound of formula 3 with protecting group, converting carbonyl group to C═S group using Lawensson's reagent, and then removing the protecting group.

The Lawensson's reagent is a common compound for converting carbonyl group to thio group and is represented as 2,4-bis(4-methoxyphenyl)-1,3-dithia-2,4-diphospetein-2,4-disulfide.

The present invention also provides a pharmaceutical composition comprising the compounds or pharmaceutically acceptable salts of formula 1 as an effective ingredient.

The compounds of the present invention inhibit in vitro binding of lck SH2 domain to specific peptide ligand. More particularly, the compounds of the present invention selectively bind the lck SH2 domain and interfere with the formation or stabilization of signaling complexes formed by proteins containing one or more SH2 domains and their natural ligands. Therefore, the compounds can be used to treat or prevent the diseases mediated by such complexes. Like this, the compounds of the present invention can be used for inhibiting the SH2-mediated cellular functions of Src based protein tyrosine kinases. The Src based protein tyrosine kinase comprises Src, Fyn, Yes, Lck, Lyn and Blk.

Also, the compounds of the present invention can be used for treating and preventing graft rejection and T cell-mediated immuno-pathological phenomena such as autoimmnune diseases by suppressing activation of T cell and their response function. Antigen-specific T cell activation may be started by TCR-mediated signal transduction process, wherein the signal transduction process is related to various tyrosine kinases, serine/threonine kinases or phsophatases. The process, which leads activated T cells to proliferation, is controlled by interaction of IL-2 with IL-2 receptor.

The present inventors performed IL-2 promoter analysis as an assay for assessing inhibitory activities of derivatives of the present invention on TCR-induced IL-2 expression. The excellent immunosuppressants should have good stability, cell permeability and must bind lck SH2 domain to suppress TCR-induced IL-2 expression.

In a preferred embodiment, by means of measuring inhibitory effect on lckSH2-pYEEI interaction and TCR-induced IL-2 expression, the present inventors confirmed that the compounds of this invention were able to pass through the cell membrane efficiently and bind lck-SH2 domain, resulting in the suppression of IL-2 gene expression and T cell proliferation, by which the pathological situation mediated by T cell was repressed (see experimental example 1 and 2).

In another preferred embodiment, for confiming the repressive effect of the expression of IL-2 gene, the present inventors carried out the standard pharmacological test in vivo by measuring the survival time of the skin allografts. In result, the present inventors confirmed that the experimental groups treated with the compounds of the present invention showed low rejection response than the control groups (see experimental example 3).

In still another preferred embodiment, to examine prophylactic or therapeutic effect on rheumatoid arthritis, one of autoimmune diseases, the present inventors measured the arthritis index in the type II collagen-induced mouse arthritis model. In result, the compounds of the present alleviated rheumatoid onset or its symptoms as effectively as Methotrexate (see experimental example 4).

Based on such results, the compounds of the present invention are useful in the treatment, diagnosis or prophylaxis of transplantation rejection such as heart, kidney, lung, liver, skin and bone marrow transplantation; autoimmune diseases such as lupus erythematous, systemic erythematous, rheumatoid arthritis, diabetes mellitus, myasthenia gravis, multiple sclerosis and psoriasis; inflammatory diseases such as dermatitis, eczema, seborrhea and inflammatory bowel; and fungal infections.

The pharmaceutical composition of the present invention may comprise pharmaceutically acceptable carriers in addition to the compounds of the present invention, and can be administered in combination with nonsteroidal anti-inflammatory agent as occasion demands. More particularly, the compounds of the present invention may also be administered in combination with one or more nonsteroidal anti-inflammatory agent for the treatment and/or prevention of organ transplantation rejection, graft-versus-host disease, autoimmune diseases and chronic inflammatory diseases in a mammal. The nonsteroidal anti-inflammatory is selected from the group consisting of aspirin, ibuprofen, naproxen, indomethacin, diclofenac, sulindac, piroxicam, etodolac, ketoprofen, meclofenamate, suprofen and tolmetin.

Administrable via oral or parenteral routes, the compounds of formula 1 can be used with oral, intravenous, subcutaneous, intranasal, intrabronchial or rectal administration, and may be used with ordinary medicine forms.

That is, the compounds of formula 1 can be formulated into various dosage forms for oral or parenteral administration. For formulation, pharmaceutically acceptable diluents, expedients and/or carriers including fillers, thickeners, binders, wetting agent, disintegration, surfactants, etc, may be used. Solid dosage forms for oral administration are exemplified by tablets, pills, powders, granules and capsules. These solid forms are prepared by admixing at least one compound of formula 1 with at least one expedient such as starch, calcium carbonate, sucrose, lactose, gelatin, etc. In addition to expedients, a lubricant such as magnesium styrate talc may be added.

Suspensions, internal solutions, emulsions, syrups, etc., are liquid dosage forms for oral administration that can comprise wetting agents, sweeteners, aromatics, and/or perspectives in addition to simple diluents such as water and liquid paraffin.

Dosage forms for parenteral administration include sterile aqueous solutions, non-aqueous solvents, suspensions, emulsions, freeze-dried agents, suppositories, etc. For formulation of non-aqueous solvents and suspensions, vegetable oils such as propylene glycol and polyethylene glycol or injectable esters such as ethyl oleate may be used. As bases for suppositories, Witepsol, macrogol, Tween 61, cocoa oil, laurinic acid and glycerogelatine are useful.

The compounds of the present invention may be administered in a dosage range of about 0.05-200 mg/kg/day when administered with intramusclular or parenteral injection, and 0.05-500 mg/kg/day when administered with oral administration.

A better understanding of the present invention may be obtained in light of the following examples which are set forth into illustrate, but are not to be construed to limit the present invention.

### EXAMPLE 1

### Preparation of 3-(3,4-dihydroxy-phenyl)-(R)-2-[3-trans-(3,4-dihydroxy-phenyl)-acryloylamino]-propionic acid methyl ester

### (step 1) Preparation of 3,4-dihydroxylphenyl-D-alanine methyl ester

In 40 ml of methanol, 2.0 g(10.14 mmol, 1 equivalent) of D-3,4-Dihydroxyphenylalanine(D-DOPA) was dissolved, and thionyl chloride(7.4 ml, 101.4 mmole, 10 equivalent) was dropwisely added to the solution at 0° C. The reaction mixture was stirred for 18 hours under nitrogen atmosphere and then distilled under a vacuo to remove excess methanol and thionyl chloride. The residue was recrystallized in methanol and ethyl acetate to provide the DOPA methyl ester. Yield was 93%.

TLC (chloroform:acetone:methanol:water=8:8:3:1); Rf=0.49

### (step 2) Preparation of 3-(3,4-dihydroxyphenyl)-(R)-2-[3-trans-(3,4-dihydroxy-phenyl)-acryloylamino]-propionic acid methyl ester

In 10 ml of N,N-dimethylformamide, 2.0 g(8.07 mmole, 1 equivalent) of DOPA methyl ester obtained in step 1 was dissolved. 1.45 g(8.07 mmole, 1 equivalent) of caffeic acid was added to the reaction mixture, and then diluted with 20 ml of methylene chloride. To the reaction mixture 4.2 g(8.07 mmole, 1 equivalent) of PyBOP and 3.4 ml of triethylamine(24.21 mmole, 3 equiv.) were added at 0° C. and then strried for 18 hours under nitrogen atmosphere. The excess methylene chloride was distilled in vacuo, diluted with 10 ml of ethyl acetate and then washed with 1 N HCl solution(3×10 ml), 10% NaHCO₃(1×10 ml), distilled water(1×10 ml) and brine(1×10 ml). The washed organic solvent was dried over anhydrous MgSO₄, filterated and concentrated in vacuo. Purification of the concentrate through flash silica gel column chromatography(normal eluent, n-hexane:ethyl acetate:methanol=4:5:1) provided the title compound. Yield was 80%.

TLC (n-hexane:ethyl acetate:methanol=4:5:1) product Rf=0.40, side product Rf=0.23 and 0.14 M/z 374 (M+H), 396 (M+Na) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂), 3.80 (3H, s, CH₃)

### EXAMPLE 2

### Preparation of 3-(3,4-dihydroxy-phenyl)-(R)-2-[3-trans-(3,4-dihydroxy-phenyl)-acryloylamino]-propionic acid

70 mg of 3-(3,4-dihydroxy-phenyl)-(R)-2-[3-trans-(3,4-dihydroxy-phenyl)-acryloylamino]-propionic acid methyl ester obtained in Example 1 was dissolved in 50 ml of mixture solvent comprising acetone and water(4:25, v/v) and then 8 ml of HCl solution was added thereto. The reaction mixture was refluxed for 1 day in oil bath, concentrated to remove acetone, and then ethyl acetate was added to obtain the title compound. The yield was 54%.

TLC(n-hexane:ethyl acetate:methanol=4:5:1) Product Rf=0.28. M/z 360.1 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂).

### EXAMPLE 3

### Preparation of 3-(3,4-dihydroxy-phenyl)-(R)-2-[3-trans-(3,4-dihydroxy-phenyl)-acryloylamino]-propionic acid ethyl ester

Except that ethanol was used as reaction solvent instead of methanol in step 1 of Example 1 to obtain D-DOPA ehtyl ester and D-DOPA ethyl ester was used as starting material, the reaction was performed in the same manner as described in Example 1, to obtain the title compound.

M/z 388.8 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.12 (2H, q, J=10.1, CH₂) 1.30 (3H, t, J=10.1, CH₃)

### EXAMPLE 4

### Preparation of 3-(3,4-dihydroxy-phenyl)-(R)-2-[3-trans-(3,4-dihydroxy-phenyl)-acryloylamino]-propionic acid propyl ester

Except n-propyl alcohol was used as reaction solvent to obtain D-DOPA propyl ester in step 1 of Example 1 and D-DOPA propyl ester was used as starting material, the reaction was performed in the same manner as described in Example 1, to obtain the title compound.

M/z 402.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.08 (2H, t, J=10.1 Hz, CH₂) 1.61 (2H, d, J=10.1, 4.90 Hz, CH₂) 0.96 (3H, t, J=10.1, 4.90 Hz, CH₃).

### EXAMPLE 5

### Preparation of 3-(3,4-dihydroxy-phenyl)-(R)-2-[3-trans-(3,4-dihydroxy-phenyl)-acryloylamino]-propionic acid isopropyl ester

Except that iso-propyl alcohol was used as reaction solvent in step 1 of Example 1 to obtain D-DOPA isopropyl ester and D-DOPA isopropyl ester was used as starting material, the reaction was performed in the same manner as described in Example 1, to obtain the title compound.

M/z 402.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.31 (1H, br, CH) 1.35 (3H,s, CH₃) 1.35 (3H, s, CH₃).

### EXAMPLE 6

### Preparation of 3-(3,4-dihydroxy-phenyl)-(R)-2-[3-trans-(3,4-dihydroxy-phenyl)-acryloylamino]propionic acid tert-butyl ester

In tetrahydrofuran the title compound of Example 2 as staring material was dissolved, and toluensulfonic acid was added to the solution. The resultant solution was reacted with isobutylene solvent, condensated using dry ice. The obtained product was seperated using HPLC having reverse column for prep to obtain the title compound.

M/z 416.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 1.40 (3H, s, CH₃) 1.40 (3H, s, CH₃)1.40 (3H, s, CH₃)

### EXAMPLE 7

### Preparation of N-[carbamoyl-2-(3,4-dihydroxy-phenyl)-ethyl]-(R)-2-[3-trans-(3,4-dihydroxy-phenyl)-acrylamide

200 g of the title compound obtained in Example 1 was dissolved in methylene chloride filled with amonia gas, and stirred for 1 day. The solution was distilled under vacuo to remove excess solvent, 30 ml of ethyl acetate was added, and washed with 1 N HCl solution(3×10 ml), 10% NaHCO₃(1×10 ml), distilled water(1×10 ml) and brine(1×10 ml). The washed organic solvent was dried with anhydrous MgSO₄, filtered, and concentrated in vacuo. Purification of the concentrate through flash silica gel column chromatography(normal eluent, n-hexane:ethyl acetate:methanol=3:5:1) provided the title compound. Yield was 80%.

TLC (n-hexane:ethyl acetate:methanol=4:5:1) Product Rf=0.30, M/z 359.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂)

### EXAMPLE 8

### Preparation of 3-(3,4-dihydroxy-phenyl)-(S)-2-[3-trans-(3,4-dihydroxy-phenyl)-acryloylamino]-propionic acid methyl ester

Except that L-DOPA was used as starting material instead of D-DOPA, the reaction was performed in the same manner as described in Example 1, to obtain the title compound.

TLC (n-hexane:ethyl acetate:methanol=4:5:1) Product Rf=0.40 M/z 374 (M+H), 396 (M+Na); ¹H NMR (DMSO-d⁶) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂), 3.80 (3H, s, CH₃)

### EXAMPLE 9

### Preparation of 3-(3,4-dihydroxy-phenyl)-(S)-2-[3-trans-(3,4-dihydroxy-phenyl)-acryloylamino]-propionic acid

Except that 3-(3,4-dihydroxy-phenyl)-(S)-2-[3-trans-(3,4-dihydroxy-phenyl)-acryloylamino]-propionic acid methyl ester obtained in Example 8 was used as starting material, the reaction was performed in the same manner as described in Example 2, to obtain the title compound.

TLC (n-hexane:ethylacetate:methanol=4:5:1) Product Rf=0.28 M/z 360.1 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂).

### EXAMPLE 10

### Preparation of 3-(3,4-dihydroxy-phenyl)-(S)-2-[3-trans-(3,4-dihydroxy-phenyl)-acryloylamino]-propionic acid ethyl ester

Except that ethanol was used as reaction solvent in step 1 of Example 1 and L-DOPA was used as starting material, the reaction was performed in the same manner as described in Example 1, to obtain the title compound.

M/z 388.8 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH₂) 4.12 (2H, q, J=10.1, CH₂) 1.30 (3H, t, J=10.1, CH₃)

### EXAMPLE 11

### Preparation of 3-(3,4-dihydroxy-phenyl)-(S)-2-[3-trans-(3,4-dihydroxy-phenyl)-acryloylamino]-propionic acid propyl ester

Except that n-propyl alcohol was used as reaction solvent in step 1 of Example 1 and L-DPRA was used as starting material, the reaction was performed in the same manner as described in Example 1, to obtain the title compound.

M/z 402.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.08 (2H, t, J=10.1 Hz, CH₂) 1.61 (2H, d, J=10.1, 4.90 Hz, CH₂) 0.96 (3H, t, J=10.1, 4.90 Hz, CH₃)

### EXAMPLE 12

### Preparation of 3-(3,4-dihydroxy-phenyl)-(S)-2-[3-trans-(3,4-dihydroxy-phenyl)-acryloylamino]-propionic acid isopropyl ester

Except that isopropyl alcohol was used as reaction solvent and L-DOPA was used as starting material in step 1 of Example 1, the reaction was performed in the same manner as described in Example 1, to obtain the title compound.

M/z 402.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.31 (1H, br, CH) 1.35 (3H,s, CH₃) 1.35 (3H, s, CH₃)

### EXAMPLE 13

### Preparation of 3-(3,4-dihydroxy-phenyl)-(S)-2-[3-trans-(3,4-dihydroxy-phenyl)-acryloylamino]-propionic acid tert-butyl ester

The title compound of (S)-form prepared in the same manner as descirbed in Example 2, as starting material, was dissolved in tetrahydrofuran and toluensulfonic acid was added thereto. The solution was reacted with isobutylene solvent, condensated by dry ice. The obtained product was seperated using HPLC having reverse column for prep to obtain the title compound.

M/z 416.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 1.40 (3H, s, CH₃) 1.40 (3H, s, CH₃)1.40 (3H, S, CH₃)

### EXAMPLE 14

### Preparation of N-[carbamoyl-2-(3,4-dihydroxy-phenyl)-ethyl]-(S)-2-[3-trans-(3,4-dihydroxy-phenyl-acrylamide

200 mg of the title compound obtained in Example 8 was dissolved in methylene chloride filled with ammonia gas, and stirred for 1 day. The solution was distilled under vacuo to remove excess solvent, dissolved in 30 ml of ethylacetate and washed with 1 N HCl solution(3×10 ml), 10% NaHCO₃ (1×10 ml), distilled water (1×10 ml), and brine (1×10 ml). The washed organic solvent was dried with MgSO₄, filtered and concentrated in vacuo. Purification of the concentrate through flash silica gel column chromatography(normal eluent, n-hexane:ethylacetate:methanol=3:5:1) provided the title compound. Yield was 80%.

TLC (n-hexane:ethylacetate:methanol=4:5:1) Product Rf=0.30 M/z 359.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂)

### EXAMPLE 15

### Preparation of 3-(3,4-dihydroxy-phenyl)-(R)-2-[3-trans-(3,4-dihydroxy-phenyl)-thioacryloylamino]-propionic acid methyl ester

### (STEP 1) Preparation of Tetra (t-butyldimethylsilyl)ether of N-(3′,4′-dihydroxy-trans-cinamoyl)-3-(3,4-dihydroxylphenyl)-D-alanine methyl ester)

In 40 ml of methylene chloride 2.44 g(6,54 mmole, 1 equiv.) of N-(3′, 4′-dihydroxy-trans-cinamoyl)-3-(3,4-dihydroxylphenyl)-D-alanine methyl ester was dissolved and 5.45 ml(39.24 mmole, 6 equiv.) of triethylamine and 7.51 ml(32.7 mmole, 5 equiv.) of t-butyldimethylsilyl trifluoromethansulfonate(TBDMSOTf) was added to the reaction mixture under nitrogen atmosphere. The reaction mixture was stirred for 18 hours under nitrogen atmosphere and 20 ml of 1N HCl solution was added at room temperature. The solution was distilled in vacuo to remove excess methylene chloride. The solution was extracted with ethylacetate(3×20 ml), and washed with distilled water(1×50 ml) and brine(1×50 ml). The residue was dried with anhydrous MgSO₄ and concentrated in vacuo. Purification of the concentrate through flash silica gel column chromatography(normal eluent, n-hexane:ethylacetate=5:1) provided the title compound. Yield was 33%.

TLC (n-hexane:ethylacetate=5:1) Product Rf=0.37 M/z 830.5 (M+H) ¹H NMR (DMSO-d⁶) δ 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H,d, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂), 3.80 (3H, s, CH₃), 0.22 (12H, s, CH₃), 0.23 (12H, s, CH₃), 0.99 (18H, s, CH₃), 1.00 (18H, s, CH₃)

### (STEP 2) Preparation of tetra(t-butyl dimethylsilyl)ether of 3-(3,4-dihydroxy-phenyl)-(R)-2-[3trans-(3,4-dihydroxy-phenyl)-thioacryloylamino]-propionic acid methyl ester

In 20 ml of tetrahydrofuran 1.8 g(2.17 mmole, 1 equiv.) of the compound obtained in step 1 was dissolved, and 1.3 g(3.25 mmole, 1.5 equiv.) of Lawensson's reagent was added. The reaction mixture was refluxed for 17 hours at 70° C., using calcium sulfate column under anhydrous state. The reactant was concentrated in vacuo and distilled with 20 ml of ethylacetate. The organic layer was added with 10% NaHCO₃ (20 ml) and the mixture was extracted with ethylacetate(3×20 ml). The extracted mixture was washed with distilled water(1×60 ml), brine(1×60 ml), dried with anhydrous MgSO₄ and concentrated in vacuo. Purification of the concentrate through flash silica gel column chromatography(normal eluent, n-hexane:ethylacetate=15:1) provided the title compound. Yield was 77%.

TLC (n-hexane:ethylacetate=15:1) Product Rf=0.40 M/z 846.4 (M+H) ¹H NMR (DMSO-d₆) δ 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H,d, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂), 3.80 (3H, s, CH₃), 0.22 (12H, s, CH₃), 0.23 (12H, s, CH₃), 0.99 (18H, s, CH₃), 1.00 (18H, s, CH₃)

### (STEP 3) Preparation of 3-(3,4-dihydroxy-phenyl)-(R)-2-[3-trans-(3,4-dihydroxy-phenyl)-thioacryloylamino]-propionic acid methyl ester

In 30 ml of THF 1.42 g(1.68 mmole, 1 equiv) of tetra(t-butyldimethylsilyl)ether-N-(3′,4′-dihydroxy-3-phenyl-2-propylenethiocarbonyl)-3-(3,4-dihydroxylphenyl)-D-alanine methyl ester was dissolved, and 1M TBAF solution(10.07 ml, 10.07 mmole, 6 equiv.) was added to the reaction mixture. The reaction mixture was stirred for 18 hours under nitrogen atmosphere, and 1N HCl solution(20 ml) was added at room temperature. The reaction mixture was concentrated in vacuo to remove excess THF and the water layer was washed with ethylacetate(3×20 ml). The extracted organic layer was washed with distilled water(1×60 ml), brine(1×60 ml), dried with anhydrous MgSO₄ and concentrated in vacuo. Purification of the concentrate through flash silica gel column chromatography(normal eluent, n-hexane:ethylacetate:methanol=4:5 1) provided the title compound. Yield was 62%.

TLC (n-hexane:ethylacetate:methanol=4:5:1) Product Rf=0.50 M/z 390.1 (M+H) ¹H NMR (DMSO-d₆) δ 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, d, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂), 3.80 (3H, s, CH₃)

### EXAMPLE 16

### Preparation of 3-(3,4-dihydroxy-phenyl)-(R)-2-[3-trans-(3,4-dihydroxy-phenyl)-thioacryloylamino]-propionic acid

3-(3,4-dihydroxy-phenyl)-(R)-2-[3-trans-(3,4-dihydroxy-phenyl)-thioacryloylamino]-propionic acid methyl ester was hydrolized in the same manner as described in Example 2, to obtain the title compound.

TLC (n-hexane:ethylacetate:methanol=4:5:1) Product Rf=0.37 M/z 376.1 (M+H);

¹H NMR (DMSO-d₆) δ 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, d, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂).

### EXAMPLE 17

### Preparation of 3-(3,4-dihydroxy-phenyl)-(R)-2-[3-trans-(3,4-dihydroxy-phenyl)-thioacryloylamino]-propionic acid ethyl ester

Except that 3-(3,4-dihydroxy-phenyl)-(R)-2-[3-trans-(3,4-dihydroxy-phenyl)-acryloylamino]-propionic acid ethyl ester obtained in Example 3 was used, the reaction was performed in the same manner as described in Example 15, to obtain the thio amide derivative.

M/z 404.1 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.12 (2H, q, J=10.1, CH₂) 1.30 (3H, t, J=10.1, CH₃)

### EXAMPLE 18

### Preparation of 3-(3,4-dihydroxy-phenyl)-(R)-2-[3-trans-(3,4-dihydroxy-phenyl)-thioacryloylamino]-propionic acid propyl ester

Except that 3-(3,4-dihydroxy-phenyl)-(R)-2-[3-trans-(3,4-dihydroxy-phenyl)-acryloylamino]-propionic acid propyl ester obtained in Example 4 was used, the reaction was performed in the same manner as described in Example 15, to obtain the thio amide derivative.

M/z 418.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.08 (2H, t, J=10.1 Hz, CH₂) 1.61 (2H, d, J=10.1, 4.90 Hz, CH₂) 0.96 (3H, t, J=10.1, 4.90 Hz, CH₃)

### EXAMPLE 19

### Preparation of 3-(3,4-dihydroxy-phenyl)-(R)-2-[3-trans-(3,4-dihydroxy-phenyl)-thioacryloylamino]-propionic acid isopropyl ester

Except that 3-(3,4-dihydroxy-phenyl)-(R)-2-[3-trans-(3,4-dihydroxy-phenyl)-acryloylamino]-propionic acid isopropyl ester obtained in Example 5 was used, the reaction was performed in the same manner as described in Example 15, to obtain the thio amide derivative.

M/z 418.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.31 (1H, br, CH) 1.35 (3H,s, CH₃) 1.35 (3H, s, CH₃)

### EXAMPLE 20

### Preparation of 3-(3,4-dihydroxy-phenyl)-(R)-2-[3-trans-(3,4-dihydroxy-phenyl)-thioacrylolamino]-propionic acid tert-butyl ester

Except that 3-(3,4-dihydroxy-phenyl)-(R)-2-[3-trans-(3,4-dihydroxy-phenyl)-acryloylamino]-propionic acid tert-butyl ester obtained in Example 6 was used, the reaction was performed in the same manner as described in Example 15, to obtain the thio amide derivative.

M/z 432.25 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 1.40 (3H, s, CH₃) 1.40 (3H, s, CH₃).

### EXAMPLE 21

### Preparation of 3-(3,4-dihydroxy-phenyl)-(R)-2-[3-trans-(3,4-dihydroxy-phenyl)-thioacryloylamino]-propioneamide

Except that N-[carbamoyl-2-(3,4-dihydroxy-phenyl)-ethyl]-(R)-2-[3-trans-(3,4-dihydroxy-phenyl)-acrylamide obtained in Example 7 was used as starting material, the reaction was performed in the same manner as described in Example 15, to obtain the title compound.

M/z 375.10 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂)

### EXAMPLE 22

### Preparation of 3-(3,4-dihydroxy-phenyl)-(S)-2-[3-trans-(3,4-dihydroxy-phenyl)-thioacryloylamino-propionic acid methyl ester

Except that 3-(3,4-dihydroxy-phenyl)-(S)-2-[3-trans-(3,4-dihydroxy-phenyl)-acryloylamino]-propionic acid methyl ester obtained in Example 8 was used as starting material, the reaction was performed in the same manner as described in Example 15, to obtain the title compound.

M/z 390.1 (M+H) ¹H NMR (DMSO-d₆) δ 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, d, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂), 3.80 (3H, s, CH₃)

### EXAMPLE 23

### Preparation of 3-(3,4-dihydroxy-phenyl)-(S)-2-[3-trans-(3,4-dihydroxy-phenyl)-thioacryloylamino]-propionic acid

Except that 3-(3,4-dihydroxy-phenyl)-(R)-2-[3-trans-(3,4-dihydroxy-phenyl)-thioacryloylamino]-propionic acid methyl ester obtained in Example 22 was used as starting material, the reaction was hydrolized in the same manner as described in Example 2 to obtain the title compound.

M/z 376.1 (M+H) ¹H NMR (DMSO-d₆) δ 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J.=1.8 Hz, aromatic), 6.61 (1H, d, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂).

### EXAMPLE 24

### Preparation of 3-(3,4-dihydroxy-phenyl)-(S)-2-[3-trans-(3,4-dihydroxy-phenyl)-thioacryloylamino]-propionic acid ethyl ester

Except that 3-(3,4-dihydroxy-phenyl)-(S)-2-[3-trans(3,4-dihydroxy-phenyl)-acryloylamino]-propionic acid ethyl ester obtained in Example 10 was used as starting material, the reaction was performed in the same manner as described in Example 15, to obtain the title compound.

M/z 404.1 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.12 (2H, q, J=10.1, CH₂) 1.30 (3H, t, J=10.1, CH₃)

### EXAMPLE 25

### Preparation of 3-(3,4-dihydroxy-phenyl)-(S)-2-[3-trans-(3,4-dihydroxy-phenyl)-thioacryloylamino]-propionic acid propyl ester

Except that 3-(3,4-dihydroxy-phenyl)-(S)-2-[3-trans-(3,4-dihydroxy-phenyl)-acryloylamino]-propionic acid propyl ester obtained in Example 11 was used as starting material, the reaction was performed in the same manner as described in Example 15 to obtain the title compound.

M/z 418.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.08 (2H, t, J=10.1 Hz, CH₂) 1.61 (2H, d, J=10.1, 4.90 Hz, CH₂) 0.96 (3H, t, J=10.1, 4.90 Hz, CH₃)

### EXAMPLE 26

### Preparation of 3-(3,4-dihydroxy-phenyl)-(S)-2-[3-trans-(3,4-dihydroxy-phenyl)-thioacryloylamino]-propionic acid isopropyl ester

Except that 3-(3,4-dihydroxy-phenyl)-(S)-2-[3-trans-(3,4-dihydroxy-phenyl)-acryloylamino]-propionic acid isopropyl ester obtained in Example 12 was used as starting material, the reaction was performed in the same manner as described in Example 15 to obtain the title compound.

M/z 418.15 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.31 (1H, br, CH) 1.35 (3H,s, CH₃).

### EXAMPLE 27

### Preparation of 3-(3,4-dihydroxy-phenyl)-(S)-2-[3-trans-(3,4-dihydroxy-phenyl)-thioacryloylamino]-propionic acid tert-butyl ester

Except that 3-(3,4-dihydroxy-phenyl)-(S)-2-[3-trans-(3,4-dihydroxy-phenyl)-acryloylamino]-propionic acid tert-butyl ester obtained in Example 13 was used as starting material, the reaction was performed in the same manner as described in Example 15 to obtain the title compound.

M/z 432.25 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 1.40 (3H, s, CH₃)

### EXAMPLE 28

### Preparation of 3-(3,4-dihydroxy-phenyl)-(S)-2-[3-trans-(3,4-dihydroxy-phenyl)-thioacryloylamino]-propioneamide

Except that N-[carbamoyl-2-(3,4-dihydroxy-phenyl)-ethyl]-(S)-2-[3-trans-(3,4-dihydroxy-phenyl)-acrylamide obtained in Example 14 was used as starting material, the reaction was performed in the same manner as described in Example 15 to obtain the title compound.

M/z 375.10 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂)

### EXAMPLE 29

### Preparation of 3-(3,4-dihydroxy-phenyl)-(R)-2-{[3-trans-(3,4-dihydroxy-phenyl)-acryloyl]-methyl-amino}-propionic acid methyl ester

### (STEP 1) Preparation of methyl N-(diphenylmethylene)-D-3-hydroxytyrosine

In 22 ml of methylene chloride 1.5 g of D-DOPA methyl ester (6.09 mmole) and 1.1 g of benzophenone imine(6.09 mmole) was dissolved, and then stirred for 24 hours at room temperature. The solution was filtered and concentrated in vacuo. The residue was dissolved with diethyl ether and filtered. The organic layer was washed with water and concentrated in vacuo. The obtained solid, was recrystallized with ethylacetate and diethyl ether to obtain the title compound as white solid. Yield 84%.

TLC (n-hexane: ethylacetate=7:3) Rf=0.2, (M+H)+375.93

In 20 ml of THF 0.76 g(2.03 mmol) of Schiff's base obtained above was dissolved. To the solution 1 M NaBH₃CN(3.2 mmol, 3.2 ml) dissolved in THF was added, and then the pH of the solution was controlled to pH 5-7 by adding acetic acid solution. After 20 minutes, 37% formaldehyde(8 mmole) and 1M NaBH₃CN dissolved in THF(12 mmole, 12 ml) were added to the solution, and then the pH of the solution was controlled to pH 5-7 by adding acetic acid solution. After 5˜6 hours, the solution was diluted with diethyl ether, washed with NaHCO₃ and brine, and concentrated. Purificaton of concentrate through silica gel column (n-hexane/ethylacetate, 6:4) provided the title compound. Yield 81%.

TLC (n-hexane/ethyl acetate, 1:1) Rf=0.7 (M+H)+391.09

### (STEP 2) Preparation of N-methyl-3,4-dihydroxy-phenyl-D-alanine methyl ester

In 17 ml of methanol 0.67 g(1.7 mmol) of the compound obtained in step 1 was dissolved. 0.07 g of Pd/C was added, and reacted under hydrogen atmosphere. After 6 hours, celite was filetered and the residue was concentrated. The concentrate was dried in vacuo and then the product was not purificated, and was used in the following reaction.

(M+H)+226.03

### (STEP 3) Preparation of 3-(3,4-dihydroxy-phenyl)-(R)-2-{[3-trans-(3,4-dihydroxy-phenyl)-acryloyl]-methyl-amino}-propionic acid methyl ester

In 13 ml of DMF N-methyl D-DOPA methyl ester obtained in step 2 and 0.3 g(1.7 mmol) of 3,4-Dihydroxy cinnamic acid were dissolved. PyBroP(2.04 mmole, 0.95 g), triethyl amine(3.4 mmole) and DMAP (1.7 mmole, 0.24 g) were added in sequence to the solution and then reacted for 5-6 horus. The reaction solution was performed in the same manner as described in Example 1 and purified using silica gel column(n-hexane:ethylacetate:methanol=4:5:1), to obtain the title compound. The purity of the compound was confirmed using RP for preparation HPLC (A is a water comprising 1% TFA, B is a acetonitrile comprising 1% TFA)(24%, 0-30% (B)/30 min, 1 ml/1 min).

TLC (n-hexane:ethylacetate:methanol=4:5:1) Rf=0.45 (M−H)−385.92, M/z 388.2 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂), 3.70 (3H, s, CH₃), 3.80 (3H, s, CH₃)

### EXAMPLE 30

### Preparation of 3-(3,4-dihydroxy-phenyl)-(R)-2-{[(3-trans-(3,4-dihydroxy-phenyl)-acryloyl-methyl-amino}-propionic acid

Except that 3-(3,4-dihydroxy-phenyl)-(R)-2-{[3-trans-(3,4-dihydroxy-phenyl)-acryloyl]-methyl-amino}-propionic acid methyl ester, obtained in Example 29, was used as starting material, the reaction was hydrolized in the same manner as described in Example 2, to obtain the title compound.

M/z 374.1 (M+H);

¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂), 3.80 (3H, s, CH₃)

### EXAMPLE 31

### Preparation of 3-(3,4-dihydroxy-phenyl)-(R)-2-{[3-trans-(3,4-dihydroxy-phenyl)-acryloyl-methyl-amino}-propionic acid ethyl ester

Except that D-DOPA ethyl ester in Example 3 was used as starting material, the reaction was performed in the same manner as described in Example 29 to obtain the title compound.

M/z 402.8 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.12 (2H, q, J=10.1, CH₂) 1.30 (3H, t, J=10.1, CH₃) 3.80 (3H, s, CH₃)

### EXAMPLE 32

### Preparation of 3-(3,4-dihydroxy-phenyl)-(R)-2-{[3-trans-(3,4-dihydroxy-phenyl)-acryloyl-methyl-amino}-propionic acid propyl ester

Except that D-DOPA propyl ester in Example 4 was used as starting material, the reaction was performed in the same manner as described in Example 29 to obtain the title compound.

M/z 416.8 (M+H) ¹H NMR (DMSO-d₆) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.08 (2H, t, J=10.1 Hz, CH₂) 1.61 (2H, d, J=10.1, 4.90 Hz, CH₂) 0.96 (3H, t, J=10.1, 4.90 Hz, CH₃) 3.80 (3H, s, CH₃)

### EXAMPLE 33

### Preparation of 3-(3,4-dihydroxy-phenyl)-(R)-2-{[3-trans-(3,4-dihydroxy-phenyl)-acryloyl]-methyl-amino}-propionic acid isopropyl ester

Except that D-DOPA isopropyl ester, obtained in Example 5, was used as starting material, the reaction was performed in the same manner as described in Example 29 to obtain the title compound.

M/z 416.8 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2) 4.31 (1H, br, CH) 1.35 (3H,s, CH3) 1.35 (3H, s, CH3) 3.80 (3H, s, CH3)

### EXAMPLE 34

### Preparation of 3-(3,4-dihydroxy-phenyl)-(R)-2-{[3-trans-(3,4-dihydroxy-phenyl)-acryloyl]-methyl-amino}-propionic acid tert-butyl ester

Except that D-DOPA tert-butyl ester, obtained in Example 6, was used as starting material, the reaction was performed in the same manner as described in Example 29 to obtain the title compound.

M/z 430.2 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2) 1.40 (3H, s, CH3) 1.40 (3H, s, CH3 )1.40 (3H, s, CH3 ) 3.80 (3H, s, CH3)

### EXAMPLE 35

### Preparation of N-[l-carbamoyl-2-(3,4-dihydroxy-phenyl)-ethyl]-(R)-3-trans-(3,4-dihydroxy-phenyl)-N-methyl-acrylamide

Except that 3-(3,4-dihydroxy-phenyl)-(R)-2-{[3-trans-(3,4-dihydroxy-phenyl)-acryloyl]-methyl-amino}-propionic acid methyl ester, obtained in Example 29, was used as starting material, the reaction was performed in the same manner as described in Example 7 to obtain the title compound.

M/z 372.15 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2), 3.80 (3H, s, CH3)

### EXAMPLE 36

### Preparation of 3-(3,4-dihydroxy-phenyl)-(S)-2-{[3-trans-(3,4-dihydroxy-phenyl)-acryloyl]-methyl-amino}-propionic acid methyl ester

Except that L-DOPA methyl ester was used as starting material, the reaction was performed in the same manner as described in Example 29 to obtain the title compound.

M/z 388.1 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2), 3.70 (3H, s, CH3), 3.80 (3H, s, CH3)

### EXAMPLE 37

### Preparation of 3-(3,4-dihydroxy-phenyl)-(S)-2-{[3-trans-(3,4-dihydroxy-phenyl)-acryloyl]-methyl-amino}-propionic acid

Except that 3-(3,4-dihydroxy-phenyl)-(S)-2-{[3-trans-(3,4-dihydroxy-phenyl)-acryloyl]-methyl-amino}-propionic acid methyl ester, obtained in Example 36, was used as starting material, the reaction was hydrolized in the same manner as described in Example 2 to obtain the title compound.

M/z 374.1 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2), 3.80 (3H, S, CH3)

### EXAMPLE 38

### Preparation of 3-(3,4-dihydroxy-phenyl)-(S)-2-{[3-trans-(3,4-dihydroxy-phenyl)-acryloyl]-methyl-amino}-propionic acid ethyl ester

Except that L-DOPA ethyl ester, obtained in the same manner as described in Example 3, was used as starting material, the reaction was performed in the same manner as described in Example 29 to obtain the title compound.

M/z 402.8 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2) 4.12 (2H, q, J=10.1, CH2) 1.30 (3H, t, J=10.1, CH3) 3.80(3H, s, CH3)

### EXAMPLE 39

### Preparation of 3-(3,4-dihydroxy-phenyl)-(S)-2-{[3-trans-(3,4-dihydroxy-phenyl)-acryloyl]-methyl-amino}-propionic acid propyl ester

Except that L-DOPA propyl ester, obtained in the same manner as described in Example 4, was used as starting material, the reaction was performed in the same manner as described in Example 29 to obtain the title compound.

M/z 416.8 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2) 4.08 (2H, t, J=10.1 Hz, CH2) 1.61 (2H, d, J=10.1, 4.90 Hz, CH2 ) 0.96 (3H, t, J=10.1, 4.90 Hz, CH3 ) 3.80 (3H, s, CH3)

### EXAMPLE 40

### Preparation of 3-(3,4-dihydroxy-phenyl)-(S)-2-{[3-trans-(3,4-dihydroxy-phenyl)-acryloyl]-methyl-amino}-propionic acid isopropyl ester

Except that L-DOPA isopropyl ester, obtained in the same manner as described in Example 5, was used as starting material, the reaction was performed in the same manner as described in Example 29 to obtain the title compound.

M/z 416.8 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2) 4.31 (1H, br, CH) 1.35 (3H,s, CH3) 1.35 (3H, s, CH3) 3.80 (3H, s, CH3)

### EXAMPLE 41

### Preparation of 3-(3,4-dihydroxy-phenyl)-(S)-2-{[3-trans-(3,4-dihydroxy-phenyl)-acryloyl]-methyl-amino}-propionic acid tert-butyl ester

Except that L-DOPA tert-butyl ester, obtained in the same manner as described in Example 6, was used as starting material, the reaction was performed in the same manner as described in Example 29 to obtain the title compound.

M/z 430.2 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2) 1.40 (3H, s, CH3) 1.40 (3H, s, CH3) 1.40 (3H, s, CH3) 3.80 (3H, s, CH3)

### EXAMPLE 42

### Preparation of N-[l-carbamoyl-2-(3,4-dihydroxy-phenyl)-ethyl]-(S)-3-trans-(3,4-dihydroxy-phenyl)-N-methyl-acrylamide

Except that 3-(3,4-dihydroxy-phenyl)-(S)-2-{[3-trans-(3,4-dihydroxy-phenyl)-acryloyl]-methyl-amino}-propionic acid methyl ester, obtained in Example 36, was used as starting material, the reaction was performed in the same manner as described in Example 7 to obtain the title compound.

M/z 372.15 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 3.29 (1H, t, J=4.9, CH2), 2.90 (1H, dd, J=13.7, 4.9 Hz, CH2), 2.72 (1H, dd, J=13.7, 4.9 Hz, CH2), 3.80 (3H, s, CH3)

### EXAMPLE 43

### Preparation of 3-(3,4-dihydroxy-phenyl)-N-[2-trans-(3,4-dihydroxy-phenyl)-ethyl]-acrylamide

Except that dopamine was used as starting material and reacted with cafeic acid, the reaction was performed with in the same manner as described in step 2 of Example 1 to obtain the title compound.

M/z 316.15 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 3.29 (2H, t, CH2) 2.90 (1H, dd, J=13.7, 4.9 Hz, CH2), 2.72 (1H, dd, J=13.7, 4.9 Hz, CH2)

### EXAMPLE 44

### Preparation of 3-(3,4-dihydroxy-phenyl)-N-[2-(3,4-dihydroxy-phenyl)-ethyl]-N-methyl-acrylamide

Except that N-methyl dopamine, obtained in the same manner as described in Example 29, was used as starting material, the reaction was performed in the same manner as described in step 2 of Example 1, to obtain the title compound.

M/z 330.15 (M+H) ¹H NMR (DMSO-d6) δ 8.62, 8.67, 9.07, 9.31 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.41 (1H, d, J=15.7 Hz, CH), 4.48 (1H, m, CH), 3.29 (2H, t, CH2) 2.90 (1H, dd, J=13.7, 4.9 Hz, CH2), 2.72 (1H, dd, J=13.7, 4.9 Hz, CH2) 3.80 (3H, S, CH3)

### EXAMPLE 45

### Preparation of (R)-2-[trans-3-(3,4-dihydroxy-phenyl)-acryloylamino]-3-(4-hydroxy-phenyl-propionic acid methyl ester

### (STEP 1) Preparation of D-tyrosine methyl ester hydrochloric acid salt

In mixture solution of thionyl chloride(27 mmol, 1.8 ml) and methanol(10 ml), D-tyrosine(2.7 mmole, 0.5 g) was dissolved at 0° C. and reacted for 15-18 hours. The reaction solution was concentrated and dried in vacuo to obtain the title compound.

### (STEP 2) Preparation of (R)-2-[trans-3-(3,4-dihydroxy-phenyl)-acryloylamino]-3-(4-hydroxy-phenyl)-propionic acid methyl ester

In 14 ml of DMF, D-tyrosine methyl ester hydrochloric acid salt obtained in step 1 was dissolved, then caffeic acid (2.9 mmole, 0.522 g), PyBOP(3.2 mmole, 1.68 g) and triethylamine(6.75 mmole, 0.94 ml) were added, and then reacted for 15-18 hours at room temperature. The solution was diluted with ethylacetate, washed with 5% HCl solution a n d brine, and concentrated. Purification of the concentrate through silica gel cloumn chromatography(n-hexane:ethylacetate:methanol=5:4:1) provided the title compound. The purity was verified using HPLC (A is a water comprising 1% TFA, B is a acetonitrile comprising 1% TFA) for RP analysis(23%, 0˜30% (B)/30min, 1 ml/1 min). The total yield: 81%

TLC (n-hexane:ethylacetate:methanol=5:4:1) Rf=0.5 M/z 358 (M+H), 370 (M+Na) ¹H NMR (DMSO-d6) δ 8.62, 8.67, 9.05 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.51 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2), 3.80 (3H, s, CH3)

### EXAMPLE 46

### Preparation of (R)-2-[trans-3-(3,4-dihydroxy-phenyl)-acryloylamino]-3-(4-hydroxy-phenyl)-propionic acid

Except that (R)-2-[trans-3-(3,4-dihydroxy-phenyl)-acryloylamino]-3-(4-hydroxy-phenyl)-propionic acid methyl ester, obtained in Example 45, was used as starting material, the reaction was performed in the same manner as described in Example 2 to obtain the title compound.

M/z 344.3 (M+H), 366.2 (M+Na) ¹H NMR (DMSO-d6) δ 8.62, 8.67, 9.05 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.51 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2)

### EXAMPLE 47

### Preparation of (R)-2-[trans-3-(3,4-dihydroxy-phenyl)-acryloylamino]-3-(4-hydroxy-phenyl)-propionic acid methyl ester

Except that L-tyrosine was used as starting material, the reaction was performed in the same manner as described in Example 45 to obtain (R)-2-[3-(3,4-dihydroxy-phenyl)-acryloylamino]-3-(4-hydroxy-phenyl)-propionic acid methyl ester(80%).

TLC (n-hexane:ethylacetate:methanol=5:4:1) Rf=0.5 M/z 358 (M+H), 370 (M+Na) ¹H NMR (DMSO-d6) δ 8.62, 8.67, 9.05 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.51 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2), 3.80 (3H, s, CH3)

### EXAMPLE 48

### Preparation of (S)-2-[trans-3-(3,4-dihydroxy-phenyl)-acryloylamino]-3-(4-hydroxy-phenyl)-propionic acid

Except that (S)-2-[trans-3-(3,4-dihydroxy-phenyl)-acryloylamino]-3-(4-hydroxy-phenyl)-propionic acid methyl ester, obtained in Example 47, was used as starting material, the reaction was performed in the same manner as described in Example 2 to obtain the title compound.

M/z 344.1 (M+H) ¹H NMR (DMSO-d6) δ 8.62, 8.67, 9.05 (4H, br, —OH), 8.75 (1H, d, J=8.0 Hz, NH), 7.20 (1H, d, J=15.7 Hz, CH), 6.93 (1H, d, J=1.8 Hz, aromatic), 6.74 (1H, d, J=8.1 Hz, aromatic), 6.62 (1H, d, J=1.8 Hz, aromatic), 6.61 (1H, J=7.8 Hz, aromatic), 6.57 (1H, d, J=7.9, 1.8 Hz, aromatic), 6.51 (1H, d, J=7.9, 1.8 Hz, aromatic), 6.41 (1H, d, J=15.7 Hz, CH), 4.48 (1H, m, CH), 2.90 (1H, dd, J=13.7, 4.9 Hz, CH2), 2.72 (1H, dd, J=13.7, 4.9 Hz, CH2)

### EXAMPLE 49

### Preparation of 2-[3-(3,4-dihydroxy-benzyl)-ureido]-3-(3,4-dihydroxy-phenyl)-propionic acid methyl ester

3,4-dimethylphenyl acetic acid (1 g, 5.1 mmol) was reacted with SOCl₂(2.65 ml) as reaction solvent. The reaction solution was concentrated in vacuo and the residue was dissolved into acetone. To the reaction solution, sodium azide solution(55.9 mmol) in 1.5 ml of distilled water was slowly added in a dropwise and then reacted for 24 hours at 0° C.

The reaction solution was concentrated in vacuo to remove the solvent. The residue was extracted with ethyl acetate, dried with MgSO₄ and then filtered. The filtered solution was concentrated in vacuo, and the residue was dissolved with 30 ml of benzene and reacted for 16 hours at 80° C. The reaction solution was concentrated under vacuo to remove the solvent and obtained the isocyanate compound. The obtained compound was dissolved in 40 ml of dichloromethane. To the solution L-DOPA methyl ester (1.717 g) solution, dissolved in dimethylformamide(2.5 ml), and triethylamine(3.38 ml) was added, and then reacted for 32 hours at room temperature. Under in vacuo, the solvent in the solution was removed to obtain 3-(3,4-dihydroxy-phenyl)-2-[3,3-dimethoxybenzylureido]propionic acid methyl ester as solid form. The obtained compound was not purified and used in the next reaction.

In the reactor maintained at −40° C., the obtained compound was introduced, and then 12.7 ml of BBR₃ solution(1 M), dissolved in dichloromethane was added. The reactor was maintained to the temperature, and the solution was reacted for 2 hours. After finishing the reaction, the temperature of the reactor was slowly increased to 4° C., and 10 ml of distilled water was introduced in the reactor. The solution was reacted for 2 hours at the same temperature, and the solvent, remained in the reactor, was removed by concentrating in vacuo. The residue was dissolved in 50 ml of ethylacetate and washed with 100 ml of water, 100 ml of brine, and then concentrated in vacuo to obtain the title compound. Yield 25%.

M/z 375.1 (M−H−) ¹H NMR(200 MHz, DMSO) δ 6.68-6.09(m, 6H), 4.40(t, 1H), 4.03(s, 2H), 3.60(s, 3H), 2.78(d, 2H)

### EXAMPLE 50

### Preparation of 3-(3,4-dihydroxy-phenyl)-2-[2-(3,4-dihydroxy-phenyl)-acetylamino]-propionic acid methyl ester

Except that 3,4-dihydroxylphenylacetic acid was used as starting material, the reaction was performed in the same manner as described in Example 1 to obtain the title compound.

M/z 360.1 (M−H−) ¹H NMR(200 MHz, DMSO) δ 6.68-6.09(m, 6H), 4.40(t, 1H), 3.93(s, 2H), 3.60(s, 3H), 2.78(d, 2H)

### EXAMPLE 51

### Preparation of 2-(3,4-dihydroxy-benzoylamino)-3-(3,4-dihydroxy-phenyl)-propionic acid methyl ester

Except that 3,4-dihydroxylbenzoic acid was used as starting material, the reaction was performed in the same manner as described in Example 1 to obtain the title compound.

M/z 346.1 (M−H−) ¹H NMR(200 MHz, DMSO) δ 6.68-6.09(m, 6H), 4.40(t, 1H), 3.60(s, 3H), 2.78(d, 2H)

### EXAMPLE 52

### Preparation of 3-(3,4-dihydroxy-phenyl)-2-[3-(3,4-dihydroxy-phenyl)-propionylamino]-propionic acid methyl ester

In the reactor, 10 g of 3-(3,4-dihydroxy-phenyl)-(R)-2-[3-trans(3,4-dihydroxy-phenyl)-acryloylamino]-propionic acid methyl ester, obtained in Example 1, was dissolved in 10 ml of methanol, and 0.3 equivalent of Pd—C was added. The solution was reacted for 18 hours at room temperature under hydrogen atmosphere. The product was treated with celite to remove impurity, and concentrated in vacuo to remove solvent. Purification of concentrate through prep-HPLC having reverse column was performed to obtain the title compound.

M/z 374.1 (M−H−) ¹H NMR(200 MHz, DMSO) δ 6.68-6.09(m, 6H), 4.40(t, 1H), 3.60(s, 3H), 2.78(d, 2H), 2.80(t, 2H), 2.47 (t, 2H)

### EXAMPLE 53

### Preparation of 3-(3,4-dihydroxy-phenyl)-2-[3-(3,4-dihydroxy-phenyl)-arylamino]-propionic acid methyl ester

In the reactor, 0.9 g(3.6 mmol, 1.3 eq.) of 3-(3,4-dihydroxy-phenyl)-(R)-2-[3-trans(3,4-dihydroxy-phenyl)-acryloylamino]-propionic acid methyl ester obtained in Example 1 was dissolved in 20 ml of THF, and 200 μl (1%) of acetic acid was added. The solution 0.5 g(2.8 mmol, 1 eq.) of 4-hydroxy-3-methoxycinamaldehyde was added to the solution, and stirred for 1 hour at room temperature. 0.53 ml(8.4 mmol, 3 eq.) of NaCNBH₃ (1M solution in THF) was slowly added to the solution and then reacted for 8 hours at room temperature.

After finishing the reaction, the solution was concentrated under vacuo to remove the solvent, and the residue was washed with ethyl acetate, 1M HCl(×2) and brine(×1). Consequently, the washed residue was dried with MgSO₄, filtered, and concentrated in vacuo. Purification of concentrate through silica gel cloumn(CHCl₃:MeOH:AcOH:H₂O=600:16:2.5:0.5) was performed to obtain 350 mg of a intermediate. Yield 33.4%.

In the reactor of −40° C., 350 mg(0.93 mmol, 1 eq) of the obtained intermediate was dissolved in 20 ml of dichloromethane. To the solution 1.6 ml(9.3 mmol, 10 eq.) of BBr₃ soution in dichloromethane(1 M) was dropwisely added and reacted for 5 hours. Consequently, temperature was slowly increased to room temperature. In the reactor, 10 ml of cooling water was introduced to finish the reaction and dichloromethane solution, remained in the reactor, was removed and washed with ethyl acetate (×2), 1M HCl(×2), brine(×1). The solution was dried with MgSO4, filtered and concentrated in vacuo. Purification of concentrate through silica gel column (CHCl₃:MeOH:AcOH:H₂O=250:16:2.5:0.5) provided the 80 mg of the title compound. Yield 24%.

M/z 358.1 (M−H−) ¹H NMR(200 MHz, DMSO) δ 6.68-6.09(m, 6H), 3.93(s, 2H), 3.60(s, 3H), 3.12 (d, 2H), 2.78(d, 2H).

### EXAMPLE 54

### Preparation of (R)-3-(3,4-dihydroxy-phenyl)-acrylic acid 2-(3,4-dihydroxy-phenyl)-1-methoxycarbonyl ethyl ester

In 10 ml of methanol as solvent, rosmarinic acid (50 mg, 0.136 mmol) was dissolved and thionyl chloride(3 eq. 98.86 mg, 72 μl) was dropwisely added at 0° C. The reaction mixture was stirred for 18 hours under nitrogen atmosphere and was distilled in vacuo to remove excess methanol and thionyl chloride. The residue was distilled in methanol. Purification of solution through prep-HPLC having reverse column was performed to obtain the title compound.

TLC (n-hexane:ethylacetate:methanol=4:5:1) Product Rf═O.69 M/z 371.15 (M+H)⁺ ¹H NMR (DMSO-d₆) δ 8.72, 8.78, 9.15, 9.64 (4H, br, —OH), 7.46 (1H, d, J=15.7 Hz, CH), 7.05 (1H, d, J=1.8 Hz, aromatic), 7.00 (1H, dd, J=8.1 Hz, 1.5 Hz aromatic), 6.76 (1H, d, J=1.8 Hz, aromatic), 6.67 (1H, J=2.0 Hz, aromatic), 6.63 (1H, J=6.2 Hz, aromatic), 6.51 (1H, dd, J=7.9, 1.8 Hz, aromatic), 6.23 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 3.19 (3H, s, CH₃).

### EXAMPLE 55

### Preparation of (R)-3-(3,4-Dihydroxy-phenyl)-acrylic acid 2-(3,4-dihydroxy-phenyl)-1-propoxycarbonyl ethyl ester

In 10 ml of 1-propyl alcohol as solvent, rosmarinic acid (50 mg, 0.136 mmol) was dissolved and thionyl chloride (3 eq. 98.86 mg, 72 μl) was dropwisely added at 0° C. The reaction mixture was stirred for 18 hours under nitrogen atmosphere and was distilled in vacuo to remove excess 1-propyl alcohol and thionyl chloride. The residue was distilled in methanol. Purification of solution through prep-HPLC having reverse column was performed to obtain the title compound.

TLC (CHCl₃:Aceton:MeOH:H₂O=8:8:3:1) Rf═0.72 M/z 403.15 (M+H)⁺ ¹H NMR (DMSO-d₆) δ 8.72, 8.78, 9.15, 9.64 (4H, br, —OH), 7.46 (1H, d, J=15.7 Hz, CH), 7.05 (1H, d, J=1.8 Hz, aromatic), 7.00 (1H, dd, J=8.1 Hz, 1.5 Hz aromatic), 6.76 (1H, d, J=1.8 Hz, aromatic), 6.67 (1H, J=2.0 Hz, aromatic), 6.63 (1H, J=6.2 Hz, aromatic), 6.51 (1H, dd, J=7.9, 1.8 Hz, aromatic), 6.23 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 3.12 (2H, t, CH₂), 1.61 (2H, m, CH₂), 1.03 (3H, t, CH₃).

### EXAMPLE 56

### Preparation of (R)-3-(3,4-Dihydroxy-phenyl)-acrylic acid 2-(3,4-dihydroxy-phenyl)-1-tert-butoxycarbonyl ethyl ester

In 5 ml of tetrahydrofuran, rosmarinic acid(100 mg, 0.278 mmol) as starting material was dissolved and 200 μl of sulfuric acid was added. The solution was reacted with isobutylene solvent, condensated using dry ice, for 3 days. Purification of solution through prep-HPLC having reverse column was performed to obtain the title compound.

TLC (n-hexane:ethylacetate:methanol=4:5:1) Product Rf=0.8 M/z 417.15 (M+H)⁺ ¹H NMR (DMSO-d₆) δ 8.72, 8.78, 9.15, 9.64 (4H, br, —OH), 7.46 (1H, d, J=15.7 Hz, CH), 7.05 (1H, d, J=1.8 Hz, aromatic), 7.00 (1H, dd, J=8.1 Hz, 1.5 Hz aromatic), 6.76 (1H, d, J=1.8 Hz, aromatic), 6.67 (1H, J=2.0 Hz, aromatic), 6.63 (1H, J=6.2 Hz, aromatic), 6.51 (1H, dd, J=7.9, 1.8 Hz, aromatic), 6.23 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 1.40 (9H, s, (CH₃)₃).

### EXAMPLE 57

### Preparation of (R)-3-(3,4-Dihydroxy-phenyl)-acrylic acid 2-(3,4-dihydroxy-phenyl)-1-carbamoyl ethyl ester

In 10 ml of tetrahydrofuran filled with ammonia gas, rosmarinic acid (50 mg, 0.136 mmol) was dissolved and stirred for 1 day. The reaction mixture was distilled to remove excess solvent and the residue was dissolved in methanol. Purification of solution through prep-HPLC having reverse column was performed to obtain the title compound.

TLC (n-hexane:ethylacetate:methanol=4:5:1) Product Rf=0.43, M/z 360.15 (M+H)⁺ ¹H NMR (DMSO-d₆) δ 8.72, 8.78, 9.15, 9.64 (4H, br, —OH), 7.46 (1H, d, J=15.7 Hz, CH), 7.05 (1H, d, J=1.8 Hz, aromatic), 7.00 (1H, dd, J=8.1 Hz, 1.5 Hz aromatic), 6.76 (1H, d, J=1.8 Hz, aromatic), 6.67 (1H, J=2.0 Hz, aromatic), 6.63 (1H, J=6.2 Hz, aromatic), 6.51 (1H, dd, J=7.9, 1.8 Hz, aromatic), 6.23 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂).

### EXAMPLE 58

### Preparation of (R)-3-(3,4-Dihydroxy-phenyl)-acrylic acid 2-(3,4-dihydroxy-phenyl)-1-isopropylcarbamoyl ethyl ester

In 10 ml of tetrahydrofuran as solvent, rosmarinic acid (100 mg, 0.278 mmol) was dissolved and thionyl chloride(3 eq. 98.86 mg, 72 μl) was dropwisely added at 0° C., 500 μl of isopropyl amine(347 mg, 5.8 mmol) was slowly added and then the solution was stirred. The reaction mixture was stirred for 18 hours under nitrogen atmosphere and was distilled in vacuo to remove excess solvent, amine and thionyl chloride. The residue was distilled in methanol. Purification of solution through prep-HPLC having reverse column was performed to obtain the title compound.

TLC (CHCl₃:Aceton:MeOH:H₂O=8:8:3:1) Rf=0.70 M/z 402.15 (M+H)⁺ ¹H NMR (DMSO-d6) δ 8.72, 8.78, 9.15, 9.64 (4H, br, —OH), 7.46 (1H, d, J=15.7 Hz, CH), 7.05 (1H, d, J=1.8 Hz, aromatic), 7.00 (1H, dd, J=8.1 Hz, 1.5 Hz aromatic), 6.76 (1H, d, J=1.8 Hz, aromatic), 6.67 (1H, J=2.0 Hz, aromatic), 6.63 (1H, J=6.2 Hz, aromatic), 6.51 (1H, dd, J=7.9, 1.8 Hz, aromatic), 6.23 (1H, d, J=15.7 Hz, CH), 4.48 (1H, m, CH), 2.90 (1H, dd, J=13.7, 4.9 Hz, CH₂), 2.72 (1H, dd, J=13.7, 4.9 Hz, CH₂) 4.31 (1H, br, CH) 1.32 (3H, s, (CH₃)₂).

The pharmaceutical composition containing the compound of formula 1 as an active ingredient can be administered orally or parenterally. The method for preparation of injection solution for parentaral administration and the method for preparation of syrup and a tablet for oral administration are illustrated as follows.

### FORMULATION EXAMPLE 1

### Preparation of an Injection solution

The injection solution containing 10 mg of the compound of formula 1 as an active ingredient was prepared as follows:

1 g of the compound of example 1, 0.6 g of NaCl and 0.1 g of ascorbic acid were dissolved in distilled water to make 100 mL of solution. The solution was filled in a bottle and sterilized by heating for 30 min at 20° C.

An injection solution of the present invention consists of the followings:

| | | |
|---|---|---|
| The compound of example 1 | 1 | g |
| NaCl | 0.6 | g |
| Ascorbic acid | 0.1 | g |

| | |
|---|---|
| Distilled water | q.s |

### FORMULATION EXAMPLE 2

### Preparation of Syrup

The syrup containing 2% (by weight/by volume) of the compound of formula 1 as an active ingredient was prepared as follows:

The compound of formula 1, saccharine, and saccharide were dissolved in 80 g of warm water. After the solution was cooled, glycerine, saccharine, flavor, ethanol, and sorbic acid were added. The distilled water was added to the mixture to make 100 mL of solution.

Syrup composition of the present invention consists of the followings:

| | | |
|---|---|---|
| The compound of example 1 | 2 | g |
| Saccharine | 0.8 | g |
| Saccharide | 25.4 | g |
| Glycerine | 8.0 | g |
| Flavor | 0.04 | g |
| Ethanol | 4.0 | g |
| Sorbic acid | 0.4 | g |

| | |
|---|---|
| Distilled water | q.s |

### FORMULATION EXAMPLE 3

### Preparation of a Tablet

The tablet containig 15 mg of the compound of formula 1 as an active ingredient was prepared as follows:

250 g of the compound of example 1 was mixed with 175.9 g of lactose, 180 g of starch and 32 g of colloidal silicic acid, and then 10% of gelatin solution was added. The resultant mixture was grounded and passed through a 14-mesh sieve and then dried. 160 g of starch, 50 g of talc and 5 g of magnesium stearate were added and blended. The resultant mixture was formulated into the tablet by conventional method.

A tablet of the present invention consists of the followings:

| | | |
|---|---|---|
| The compound of example 1 | 250 | g |
| Lactose | 175.9 | g |
| Starch | 180 | g |
| Colloidal silicic acid | 32 | g |
| 10% of gelatin solution | 160 | g |
| Talc | 50 | g |
| Magnesium stearate | 5 | g |

The following experimental examples illustrate the efficacy of the compounds, prepared by example 1-53, as inhibitor of lck SH2 domain or inhibition of IL-2 gene expression, to result in T cell proliferation inhibition.

### EXPERIMENTAL EXAMPLE 1

### Inhibition of the Compounds of the Present Invention on in vitro Binding of lck SH2 Domain and its Cognate Peptide

The present inventors investigated the inhibition activity of phenyl derivatives on the interaction of GST (Glutathione transferase)-fused lck SH2 domain and a peptide SGSGEEPQpYEEIPI (comprising the sequence PYEEI, a cognate peptide of Lck SH2) by using a real time sensorgram (BIAcore 2000).

At first, the biotinylated peptide was fixed on the surface of BIAcore analysis apparatus and then GST-lckSH2 protein was injected over the surface of the immobilized peptides. The binding of GST-lck SH2 with the peptide is represented as the resonance units (referred as “RU” hereinafter) and 1,000 RU corresponds to a change in surface concentration of 1 ng/mm². The binding of GST-lck with the fixed peptide causes the change of refractory index, leading to the increase of RU. In the mean time, when the binding of GST-lck with the fixed peptide is inhibited, RU decreases. Those results are represented in Table 1. As seen in Table 1, the capacity of the compound of the invention to inhibit the binding between the fixed peptide and GST-lckSH2 is represented by the IC₅₀ (50% inhibition concentration), wherein +: 25-50 uM; ++: 10-25uM; and +++: <10 uM.

### TABLE 1

The inhibitory effect of the phenyl derivatives on lck SH2-PYEEI interaction

| | | BOND DEGREE |
|---|---|---|
| | | WITH SH2 |
| EX | STRUCTURE | OF 1ck |
| 1 | 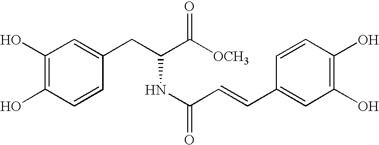 | +++ |
| 2 | 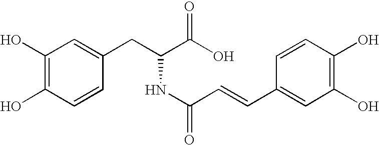 | +++ |
| 3 | 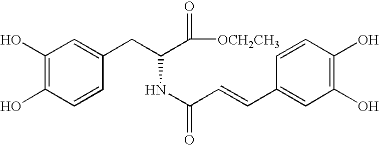 | +++ |
| 4 | 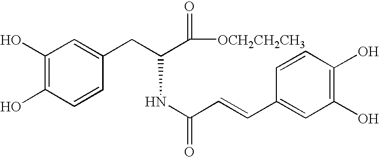 | +++ |
| 5 | 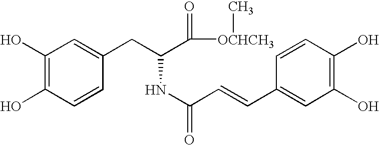 | +++ |
| 6 | 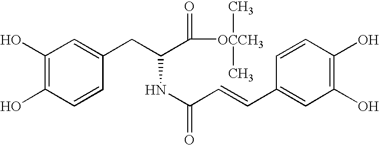 | ++ |
| 7 | 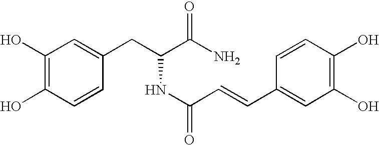 | +++ |
| 8 | 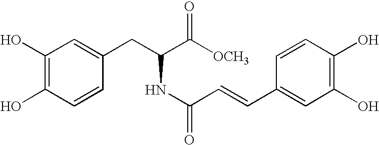 | ++ |
| 9 | 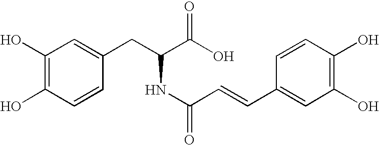 | + |
| 10 | 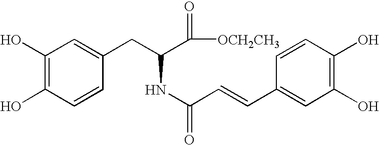 | ++ |
| 11 | 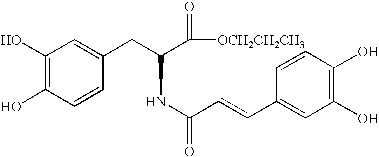 | ++ |
| 12 | 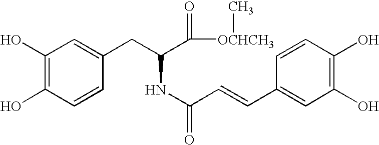 | ++ |
| 13 | 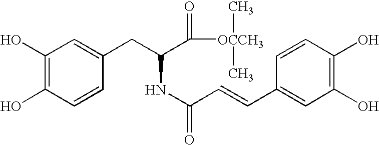 | + |
| 14 | 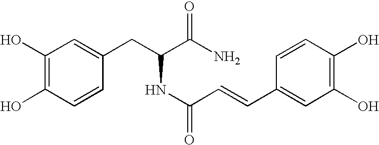 | ++ |
| 15 | 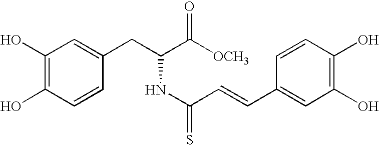 | ++ |
| 16 | 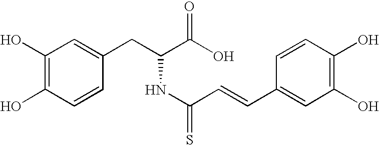 | + |
| 17 | 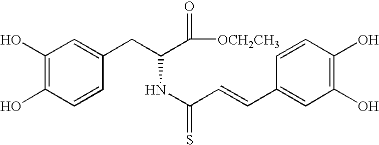 | ++ |
| 18 | 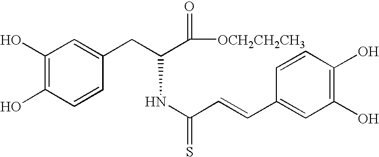 | ++ |
| 19 | 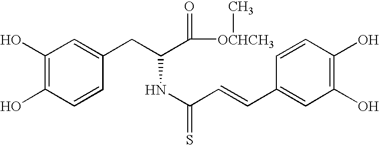 | ++ |
| 20 | 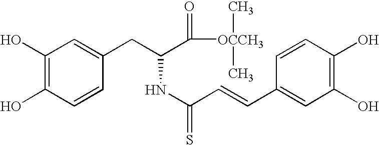 | + |
| 21 | 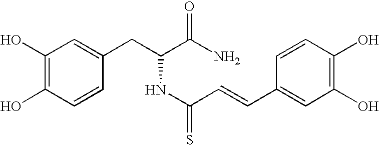 | ++ |
| 22 | 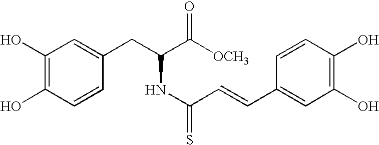 | + |
| 23 | 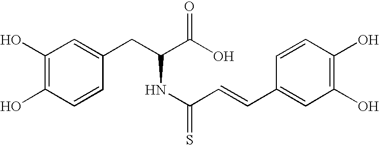 | + |
| 24 | 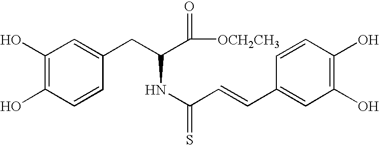 | ++ |
| 25 | 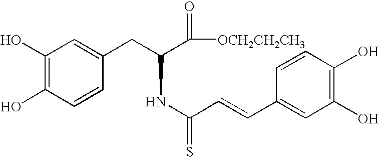 | ++ |
| 26 | 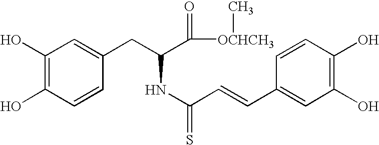 | + |
| 27 | 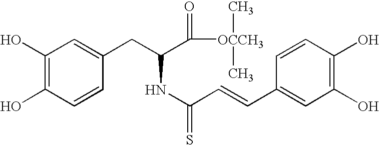 | + |
| 28 | 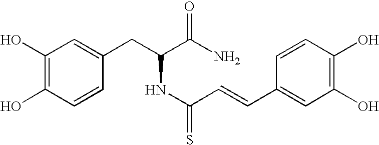 | ++ |
| 29 | 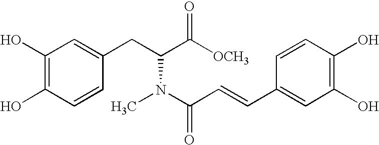 | ++ |
| 30 | 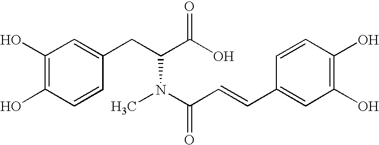 | + |
| 31 | 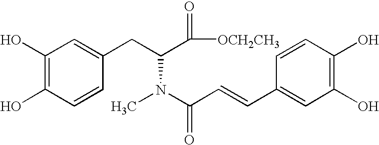 | ++ |
| 32 | 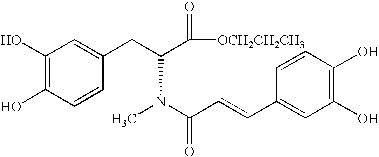 | ++ |
| 33 | 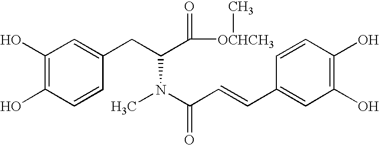 | ++ |
| 34 | 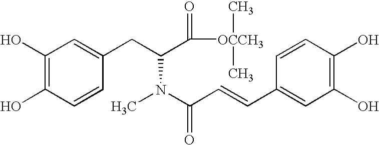 | + |
| 35 | 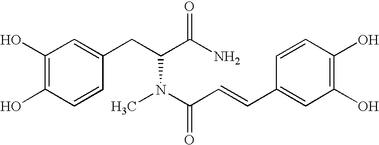 | ++ |
| 36 | 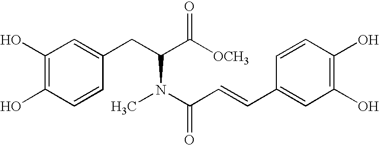 | + |
| 37 | 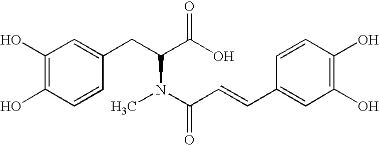 | + |
| 38 | 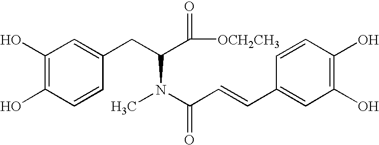 | ++ |
| 39 | 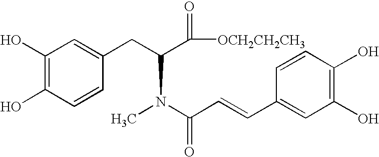 | ++ |
| 40 | 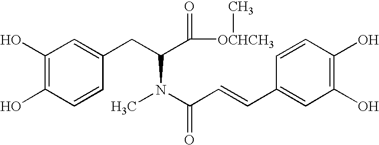 | ++ |
| 41 | 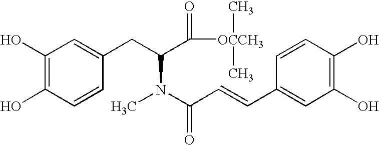 | + |
| 42 | 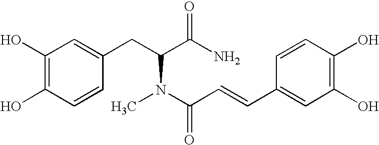 | ++ |
| 43 | 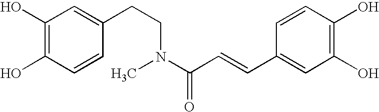 | + |
| 44 | 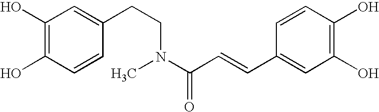 | + |
| 45 | 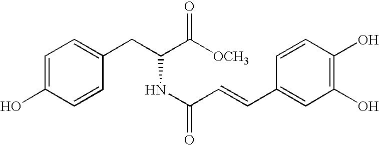 | ++ |
| 46 | 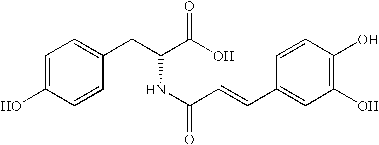 | + |
| 47 | 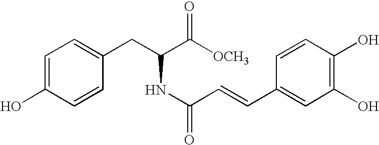 | + |
| 48 | 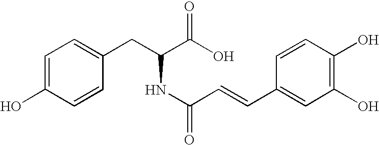 | + |
| 49 | 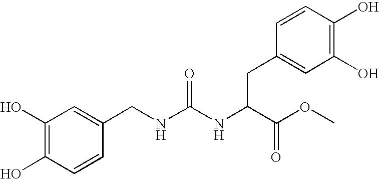 | ++ |
| 50 | 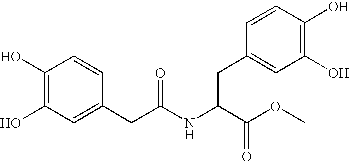 | +++ |
| 51 | 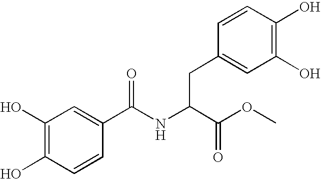 | +++ |
| 52 | 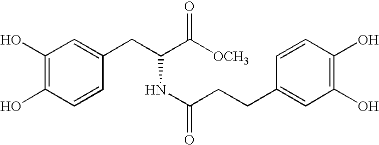 | ++ |
| 53 | 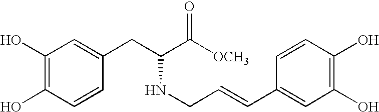 | + |
| 54 | 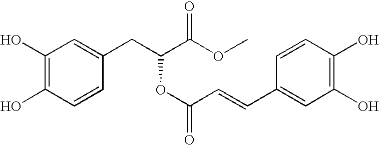 | ++ |
| 55 | 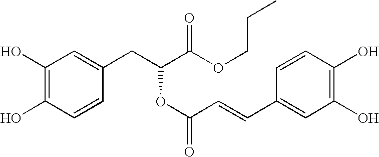 | ++ |
| 56 | 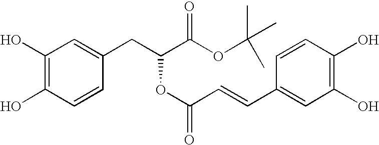 | + |
| 57 | 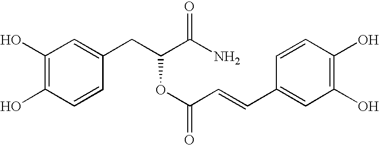 | + |
| 58 | 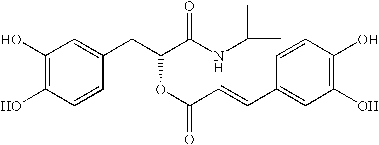 | + |

+: 25˜50 μM;
++: 10˜25 μM;
+++: below 10 μM

As shown in Table 1, the inhibitory effect of the compounds of the present invention on lck SH2-pYEEI interaction is most efficient when Y₁ and Y₂ of the compounds of the invention are to be a part of forming ester or amide and so is when X₁ is —NH— or —O—; X₂ is —C(═O)— or —C(═S)—; and X₃ is

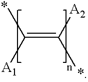

The compounds of this invention with high affinity to GST-lckSH2 can be used for the prevention or for the treatment of T cell disorder-oriented diseases such as autoimmune diseases, T cell leukemia or allograft rejection processes by inhibiting lckSH2-mediated signaling leading to T cell activation and proliferation.

### EXPERIMENTAL EXAMPLE 2

### Inhibition of IL-2 Gene Expression

In order to confirm whether the compounds of the invention can pass through a cell membrane and inhibit the expression of IL-2 gene leading to T-cell proliferation, the present inventors firstly confirmed T-cell activity by detecting the activity of the luciferase fused on the downstream of IL-2 promoter.

To investigate the activity of IL-2 promoter, Jurkat cells (1×10⁶) were transfected with a IL-2 reporter plasmids using Superfect™ (Qiagen Inc.). After 24 hours of transfection, Jurkat cells were pre-incubated with compounds of various concentrations (1 uM-50 uM) for 2 hours and further cultured on 35mm plate pre-coated with 5 ug/ml of anti-CD3 antibody, resulting in T cell activation. The luciferase activity was measured using Berthold luminometer LB953. The IC₅₀ (50% inhibition concentration) of the compounds of the invention on TCR-induced IL-2 promoter activation are shown in Table 2.

TABLE 2

| | Activity of IL- |
|---|---|
| | 2 promoter |
| EXAMPLE | IC₅₀ (μM) |
| 1 | 1.2 |
| 2 | 15 |
| 3 | 4.8 |
| 4 | 7.8 |
| 5 | 6.5 |
| 6 | 7.8 |
| 7 | 2.4 |
| 8 | 10.9 |
| 9 | 18.2 |
| 10 | 4.4 |
| 11 | 4.9 |
| 12 | 9.5 |
| 13 | 7.2 |
| 14 | 6.9 |
| 15 | 17.1 |
| 16 | 6.7 |
| 17 | 4.6 |
| 18 | 5.6 |
| 19 | 8.9 |
| 20 | 21.4 |
| 21 | 7.8 |
| 22 | 13.9 |
| 23 | 16.0 |
| 24 | 10.8 |
| 25 | 9.7 |
| 26 | 18.7 |
| 27 | 20.1 |
| 28 | 14.1 |
| 29 | 7.6 |
| 30 | 15.8 |
| 31 | 8.9 |
| 32 | 9.0 |
| 33 | 7.8 |
| 34 | 14.9 |
| 35 | 9.7 |
| 36 | 15.0 |
| 37 | 17.3 |
| 38 | 11.0 |
| 39 | 9.7 |
| 40 | 8.7 |
| 41 | 15.7 |
| 42 | 14.6 |
| 43 | 21.2 |
| 44 | 17.4 |
| 45 | 7.8 |
| 46 | 19.0 |
| 47 | 21.0 |
| 48 | 22.3 |
| 49 | 20.4 |
| 50 | 18.6 |
| 51 | 17.5 |
| 52 | 21.0 |
| 53 | 24.4 |
| 54 | 4.5 |
| 55 | 5.7 |
| 56 | 6.2 |
| 57 | 5.2 |
| 58 | 5.4 |

As shown in table 2, the compounds of the invention effectively inhibited TCR-induced IL-2 promoter activation with IC₅₀ over the range from 1 uM to 25 uM. Particularly, the compounds of example 1, 3, 7, 10, 11, 17 and 54-58 strongly inhibited IL-2 promoter activation.

As explained hereinbefore, the compounds of the invention can be effectively used for the prevention or the treatment of T-cell mediated diseases such as autoimmune or chronic inflammatory diseases by inhibiting TCR-induced signaling leading to T-cell activation and proliferation.

### EXPERIMENTAL EXAMPLE 3

### Inhibition of the Skin Allograft Rejection

The present inventors confirmed the inhibitory effect of phenyl derivatives of the invention on allograft rejection by measuring the survival time of the grafted skin on test animals after skin allograft transplantation using mouse model.

For the experiment, allogeneic Balb/c(H-2d) mouse-tail skin was grafted onto C57BL/6(H-2b) mouse. Generally 7-8 mice were assigned to each group. All the phenyl derivatives were dissolved in 100% ethanol, mixed with olive oil, and adjusted to the final concentration of ethanol therein less than 5%. The compounds of the invention (100 mg/kg/day) were administered directly into abdominal cavity of the test animals from the day of transplantation until the complete rejection occurred. Meanwhile, for the group of untreated mice, an olive oil-ethanol mixture was administered as a control. The grafted skin was monitored daily after the removal of the bandage on days 7-9. Rejection was defined as more than 80% necrosis of the graft epithelium. The test results were shown in Table 3.

TABLE 3
Inhibition of the skin allograft rejection

| | Survival time of | | Survival time of |
|---|---|---|---|
| | skin graft tissue | | skin graft tissue |
| Example | (days ± variance) | Example | (days ± variance) |
| 1 | 15.1 ± 1.5 | 42 | 14.3 ± 0.9 |
| 2 | 14.8 ± 0.5 | 43 | 13.1 ± 0.9 |
| 8 | 15.2 ± 1.1 | 44 | 12.5 ± 0.9 |
| 9 | 14.8 ± 0.9 | 46 | 11.9 ± 0.9 |
| 10 | 15.3 ± 0.8 | 47 | 11.7 ± 1.0 |
| 11 | 14.4 ± 0.9 | 48 | 12 ± 1.6 |
| 12 | 15.7 ± 0.8 | 49 | 13 ± 1.5 |
| 13 | 14.5 ± 0.6 | 50 | 12.8 ± 0.9 |
| 14 | 15.3 ± 0.9 | 51 | 12.5 ± 1.1 |
| 23 | 13.2 ± 0.8 | 52 | 13.4 ± 1.2 |
| 25 | 14.8 ± 0.6 | 53 | 11.9 ± 1.1 |
| 27 | 12.3 ± 0.6 | 54 | 13 ± 0.9 |
| 28 | 13.9 ± 0.9 | 55 | 12.1 ± 0.8 |
| 36 | 12.9 ± 0.9 | 56 | 12.9 ± 1.2 |
| 39 | 14.1 ± 1.2 | 57 | 13.1 ± 0.7 |
| 41 | 13.7 ± 0.6 | 58 | 12.5 ± 1.3 |
| Prepara- | 10.5 ± 0.9 | Cyclo- | 13 ± 1.4 |
| tive ex | | sporine A | |

| | |
|---|---|
| Rapamycine 4 mg/kg/day | 16.2 ± 1.0 |
| Rapamycine 1 mg/kg/day | 12.7 ± 1.5 |
| Rapamycine 1 mg/kg/day + | 18.3 ± 1.3 |
| Example (1) 50 mg/kg/day | |
| Example (1) 50 mg/kg/day | 11.2 ± 0.5 |
| Control | 10.3 ± 0.5 |

As shown in table 3, the effects of the compounds of resent invention to prevent rejection after skin aft were monitored. Particularly, on day 9-10 when ion was observed in the vehicle control group, the ion process was not seen in the experimental group d with the compounds of the invention, or, if any, rejection like black-wrinkled scabs were observed in than 20% of skin grafted tissues. In addition, the d skin survived 3-5 days longer in the group treated he compounds of the invention.

When 50 mg/kg/day (suboptimal dose) of the compounds of the example 1 was administered together with 4 mg/kg/day (optimal dose) of Rapamycin, a classical immunosuppressive drug, immunosuppressive effect was augmented greatly compared to administrating Rapamycin only.

The compounds of the invention, thus, can be effectively used for the prevention or the treatment of T-cell mediated diseases such as transplantation rejection by inhibiting TCR-induced T-cell activation and proliferation.

### EXPERIMENTAL EXAMPLE 4

### Inhibition of Collagen-Induced Rheumatoid Arthritis

The present inventors experimentally induced rheumatoid arthritis by injecting DBA1/LacJ mice with emulsions of 100 μg of bovine type II collagen(C II) and complete Freund's Adjuvant (CFA) hypodermically into the tail base (male, 8 weeks). After 2 weeks, booster immunization was carried out with 50 μg C II/IFA. From the 3^{rd} week after primary immunization, compounds of the present invention were injected daily into peritoneal cavities of 6-8 mice per group for 15-20 days.

The vehicle control groups were injected daily with 100 μl of 5% ethanol-olive oil mixture in total 15-20 times. Widely used anti-rheumatic drug, Methotrexate, was dissolved in PBS and then injected to mice at 1 mg/kg/day every other day in total 8-10 times. Meanwhile, the compounds of the invention were dissolved in ethanol and then mulsified in olive oil, in which the final ethanol concentration was adjusted to 5% and the compounds of this invention was 50 mg/kg/day.

From the 3^{rd} week after primary immunization, the extent of edema and the joint swelling was monitored everyday to determine arthritic index. Arthritic score was determined based on the criteria listed in Table 4. Arthitic index is the sum of the arthritic scores of all 4 legs and, therefore, maximum arthritis index of 16 can be achieved (e.g., [4 (maximum arthritic score)/leg]×[4 legs/mouse]=16 (maximum arthritis index/mouse)). The of each compound to inhibit collagen-induced tis was presented by the mean arthritis index±standard deviation of each mouse group. The results were presented in Table 5.

TABLE 4

| Arthritis | |
|---|---|
| score | Symptoms |
| 0 | No swelling and boil |
| 1 | A mild swelling and reddened localized in mid |
| | foot(tarsal bone) or angle joint |
| 2 | A mild swelling and reddened foot from angle |
| | joint to mid foot |
| 3 | A medium swelling and reddened foot from angle |
| | joint to metatarsal bone |
| 4 | A swelling and reddened foot covering the |
| | whole from angle to digit |

TABLE 5
Inhibition of arthritis

| Example | Arthritis index* | Example | Arthritis index* |
|---|---|---|---|
| 1 | 4.3 ± 1.5 | 31 | 5.9 ± 2.1 |
| 2 | 7.2 ± 1.5 | 32 | 5.3 ± 1.7 |
| 3 | 4.5 ± 0.8 | 33 | 5.7 ± 0.9 |
| 4 | 5.6 ± 0.5 | 34 | 6.3 ± 0.8 |
| 5 | 4.9 ± 1.2 | 35 | 6.8 ± 1.9 |
| 6 | 5.8 ± 0.9 | 36 | 6.1 ± 1.9 |
| 7 | 4.3 ± 1.5 | 37 | 6.7 ± 0.7 |
| 8 | 6.0 ± 1.1 | 38 | 5.5 ± 1.3 |
| 9 | 7.2 ± 1.2 | 39 | 5.7 ± 1.2 |
| 10 | 5.8 ± 0.8 | 40 | 5.9 ± 0.4 |
| 11 | 6.1 ± 1.3 | 41 | 6.7 ± 1.6 |
| 12 | 5.1 ± 0.7 | 42 | 5.1 ± 1.9 |
| 13 | 6.9 ± 1.1 | 43 | 6.0 ± 1.9 |
| 14 | 5.3 ± 1.2 | 44 | 6.8 ± 1.0 |
| 15 | 5.7 ± 0.9 | 45 | 5.7 ± 0.9 |
| 16 | 7.2 ± 1.2 | 46 | 6.1 ± 0.7 |
| 17 | 5.4 ± 0.7 | 47 | 6.9 ± 1.1 |
| 18 | 5.8 ± 0.3 | 48 | 7.1 ± 0.4 |
| 19 | 5.1 ± 1.1 | 49 | 5.1 ± 0.9 |
| 20 | 6.7 ± 0.4 | 50 | 4.8 ± 1.1 |
| 21 | 5.8 ± 1.3 | 51 | 5.5 ± 0.5 |
| 22 | 6.9 ± 1.2 | 52 | 4.0 ± 0.7 |
| 23 | 6.7 ± 0.8 | 53 | 9.8 ± 1.2 |
| 24 | 6.1 ± 0.6 | 54 | 3.5 ± 1.1 |
| 25 | 5.7 ± 0.4 | 55 | 4.8 ± 0.8 |
| 26 | 7.0 ± 0.9 | 56 | 5.2 ± 0.5 |
| 27 | 6.6 ± 1.1 | 57 | 4.5 ± 1.2 |
| 28 | 5.8 ± 0.6 | 58 | 4.8 ± 0.7 |
| 29 | 6.1 ± 2.1 | | |
| 30 | 6.5 ± 0.6 | | |
| Compar- | 14.0 ± 2.0 | MTX | 4.0 ± 1.1 |
| ative ex. | | | |

*Arthritis index represents the sum of arthritis scores of all four legs (maximum 16 point). The arthritis indexes listed above were obtained on day 15 after treatment of drug or compounds of this invention (day 36 after primary immunization).
MTX: methotrexate

As shown in table 5, hydroxyl phenyl derivatives were very effective for the treatment of arthritis on the whole.

As IL-2 promoter assay illustrated by the above example, hydroxyl phenyl derivatives were proved to be more effective when methyl and ethyl residues were added to carboxyl residue, which might be due to improved cell permeability resulting from increasing hydrophobicity of the compounds. There was no big difference in binding assay, though.

### EXPERIMENTAL EXAMPLE 5

### Acute Oral Toxicity Test in Rats

The following experiments were performed to determine acute toxicity of compounds of this invention in rats.

6-week old SPF SD line rats were used in determining acute toxicity. The compounds of examples were suspended in 0.5% methylcellulose solution and orally administered once to 2 rats per group at the dosage of 1 g/kg/15 ml. Death, clinical symptoms, and weight change in rats were observed, hematological tests and biochemical tests of blood were performed, and any abnormal signs in the gastrointestinal organs of chest and abdomen were checked with eyes during autopsy. The results showed that the test compounds did not cause any specific clinical symptoms, weight change, or death in rats. No change was observed in hematological tests, biochemical tests of blood, and autopsy. As a result, the compounds used in this experiment are evaluated to be safe substances since they do not cause any toxic change in rats up to the level of 500 mg/kg and their estimated LD₅₀ values are much greater than 1 g/kg in rats. Thus, the compounds of the present invention represented by formula 1 were proved to be safe compounds for intravenous, subcutaneous, intranasal, intrabronchial, rectal and oral administration.

As explained hereinbefore, the compounds of the present invention inhibited the molecular interactions of lckSH2 and its cognate peptide PYEEI and TCR-induced IL-2 gene expression, resulting in immunosuppression in vitro and in vivo. Therefore, the compounds of the invention can be effectively used for inhibiting lck SH2 domain or Src based protein tyrosine kinase SH2 domain and suppressing allograft rejection, autoimmune diseases and inflammatory diseases. Also, the compounds of the present invention have sufficiently high activity at low dosages and low side effects being observed in currently used therapeutics for arthritis, so that they can be used for the treatment or prevention of rheumatoid arthritis or inflammatory diseases.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. Derivatives of formula 1, or pharmaceutically acceptable salts thereof. 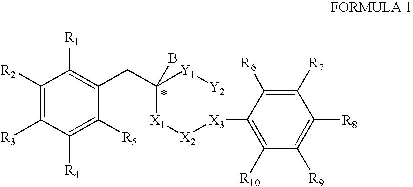
Wherein, R₁, R₂, R₃, R₄ and R₅ are all independent of each other and at least one of them is hydroxyl group, others are selected from the group consisting of hydrogen; halogen atom; C₁˜C₃ alkoxy; aldehyde; carboxyl; amino; trifluoromethyl; and nitro;
R₆, R₇, R₈, R₉ and R₁₀ are also independent of each other and at least one of them is hydroxy group, others are selected from the group consisting of hydrogen; halogen atom; C₁˜C₃ alkoxy; aldehyde; carboxyl; amino; trifluoromethyl; and nitro;
X₁ is O; S; —NH; —N(CH₃)—; —N(CH₂CH₃)—; or —NHNH—;
X₂ is —CH₂—; —C(═O)—; —C(═S)—; or —C(═O)—NH—;
X₃ is selected from the group consisting of 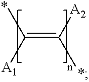 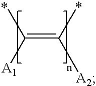
and —(CH₂)ₘ—;
wherein A₁ is hydrogen; C₁˜C₄ straight or branched alkyl; thiol; phenyl; cyano; or C₁˜C₃ alkoxycarbonyl,
A₂ is hydrogen; or C₁˜C₄ straight or branched alkyl, n is 0, 1 or 2, m is 0, 1 or 2;
Y₁ is selected from the group consisting of hydrogen; —CH₂—; —C(═O)—; —C(═S)—; C₁˜C₄ straight or branched alkyl or amine substituted with aryl; and 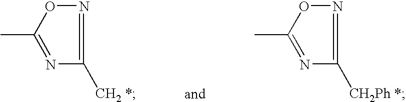
Y₂ does not exist or is —NZ₁₁Z₁₂; —O—Z₂; or —S-Z₂;
wherein Z₁₁ and Z₁₂ are independent each other and can be hydrogen; amine optionally substituted with t-butoxycarbonyl; C₁˜C₁₂ straight or branched alkyl; aryl; cycloalkyl; or heteroalkyl;
Z₂ is hydrogen; C₁˜C₁₂ straight or branched alkyl; aryl; cycloalkyl; or heteroalkyl;
B is hydrogen or alkyl;
* represents a chiral carbon.

2. The derivatives or salts of thereof according to claim 1, wherein Y₁ and Y₂ are bonded, C₁-C₅ straight or branched alkoxycarbonyl or amide.

3. The derivatives or salts thereof according to claim 1, wherein the derivatives are selected from the group consisting of:
1) 3-(3,4-dihydroxy-phenyl)-(R)-2-[3-trans-(3,4-dihydroxy-phenyl)-acryloylamino]-propionic acid methyl ester;
2) 3-(3,4-dihydroxy-phenyl)-(R)-2-[3-trans-(3,4-dihydroxy-phenyl)-acryloylamino]-propionic acid;
3) 3-(3,4-dihydroxy-phenyl)-(R)-2-[3-trans-(3,4-dihydroxy-phenyl)-acryloylamino]-propionic acid ethyl ester;
4) 3-(3,4-dihydroxy-phenyl)-(R)-2-[3-trans-(3,4-dihydroxy-phenyl)-acryloylamino]-propionic acid propyl ester;
5) 3-(3,4-dihydroxy-phenyl)-(R)-2-[3-trans-(3,4-dihydroxy-phenyl)-acryloylamino]-propionic acid isopropyl ester;
6) 3-(3,4-dihydroxy-phenyl)-(R)-2-[3-trans-(3,4-dihydroxy-phenyl)-acryloylamino]-propionic acid tert-butyl ester;
7) N-[carbamoyl-2-(3,4-dihydroxy-phenyl)-ethyl]-(R)-2-[3-trans-(3,4-dihydroxy-phenyl)-acrylamide;
8) 3-(3,4-dihydroxy-phenyl)-(S)-2-[3-trans-(3,4-dihydroxy-phenyl)-acryloylamino]-propionic acid methyl ester;
9) 3-(3,4-dihydroxy-phenyl)-(S)-2-[3-trans-(3,4-dihydroxy-phenyl)-acryloylamino]-propionic acid;
10) 3-(3,4-dihydroxy-phenyl)-(S)-2-[3-trans-(3,4-dihydroxy-phenyl)-acryloylamino]-propionic acid ethyl ester;
11) 3-(3,4-dihydroxy-phenyl)-(S)-2-[3-trans-(3,4-dihydroxy-phenyl)-acryloylamino]-propionic acid propyl ester;
12) 3-(3,4-dihydroxy-phenyl)-(S)-2-[3-trans-(3,4-dihydroxy-phenyl)-acryloylamino]-propionic acid isopropyl ester;
13) 3-(3,4-dihydroxy-phenyl)-(S)-2-[3-trans-(3,4-dihydroxy-phenyl)-acryloylamino]-propionic acid tert-butyl ester;
14) N-[carbamoyl-2-(3,4-dihydroxy-phenyl)-ethyl]-(S)2-[3-trans-(3,4-dihydroxy-phenyl)-acrylamide;
15) 3-(3,4-dihydroxy-phenyl)-(R)-2-[3-trans-(3,4-dihydroxy-phenyl)-thioacryloylamino]-propionic acid methyl ester;
16) 3-(3,4-dihydroxy-phenyl)-(R)-2-[3-trans-(3,4-dihydroxy-phenyl)-thioacryloylamino]-propionic acid;
17) 3-(3,4-dihydroxy-phenyl)-(R)-2-[3-trans-(3,4-dihydroxy-phenyl)-thioacryloylamino]-propionic acid ethyl ester;
18) 3-(3,4-dihydroxy-phenyl)-(R)-2-[3-trans-(3,4-dihydroxy-phenyl)-thioacryloylamino]-propionic acid propyl ester;
19) 3-(3,4-dihydroxy-phenyl)-(R)-2-[3-trans-(3,4-dihydroxy-phenyl)-thioacryloylamino]-propionic acid isopropyl ester;
20) 3-(3,4-dihydroxy-phenyl)-(R)-2-[3-trans-(3,4-dihydroxy-phenyl)-thioacryloylamino]-propionic acid tert-butyl ester;
21) 3-(3,4-dihydroxy-phenyl)-(R)-2-[3-trans-(3,4-dihydroxy-phenyl)-thioacryloylamino]-propioneamide;
22) 3-(3,4-dihydroxy-phenyl)-(S)-2-[3-trans-(3,4-dihydroxy-phenyl)-thioacryloylamino]-propionic acid methyl ester;
23) 3-(3,4-dihydroxy-phenyl)-(S)-2-[3-trans-(3,4-dihydroxy-phenyl)-thioacryloylamino]-propionic acid;
24) 3-(3,4-dihydroxy-phenyl)-(S)-2-[3-trans-(3,4-dihydroxy-phenyl)-thioacryloylamino]-propionic acid ethyl ester;
25) 3-(3,4-dihydroxy-phenyl)-(S)-2-[3-trans-(3,4-dihydroxy-phenyl)-thioacryloylamino]-propionic acid propyl ester;
26) 3-(3,4-dihydroxy-phenyl)-(S)-2-[3-trans-(3,4-dihydroxy-phenyl)-thioacryloylamino]-propionic acid isopropyl ester;
27) 3-(3,4-dihydroxy-phenyl)-(S)-2-[3-trans-(3,4-dihydroxy-phenyl)-thioacryloylamino]-propionic acid tert-butyl ester;
28) 3-(3,4-dihydroxy-phenyl)-(S)-2-[3-trans-(3,4-dihydroxy-phenyl)-thioacryloylamino]-propioneamide;
29) 3-(3,4-dihydroxy-phenyl)-(R)-2-{[3-trans-(3,4dihydroxy-phenyl)-acryloyl]-methyl-amino}-propionic acid methyl ester;
30) 3-(3,4-dihydroxy-phenyl)-(R)-2-{[3-trans-(3,4dihydroxy-phenyl)-acryloyl]-methyl-amino}-propionic acid;
31) 3-(3,4-dihydroxy-phenyl)-(R)-2-{[3-trans-(3,4dihydroxy-phenyl)-acryloyl]-methyl-amino}-propionic acid ethyl ester;
32) 3-(3,4-dihydroxy-phenyl)-(R)-2-{[3-trans-(3,4dihydroxy-phenyl)-acryloyl]-methyl-amino}-propionic acid propyl ester;
33) 3-(3,4-dihydroxy-phenyl)-(R)-2-{[3-trans-(3,4dihydroxy-phenyl)-acryloyl]-methyl-amino}-propionic acid isopropyl ester;
34) 3-(3,4-dihydroxy-phenyl)-(R)-2-{[3-trans-(3,4dihydroxy-phenyl)-acryloyl]-methyl-amino}-propionic acid tert-butyl ester;
35) N-[1-carbamoyl-2-(3,4-dihydroxy-phenyl)-ethyl](R)-3-trans-(3,4-dihydroxy-phenyl)-N-methyl-acrylamide;
36) 3-(3,4-dihydroxy-phenyl)-(S)-2-{[3-trans-(3,4dihydroxy-phenyl)-acryloyl]-methyl-amino}-propionic acid methyl ester;
37) 3-(3,4-dihydroxy-phenyl)-(S)-2-{[3-trans-(3,4dihydroxy-phenyl)-acryloyl]-methyl-amino}-propionic acid;
38) 3-(3,4-dihydroxy-phenyl)-(S)-2-{[3-trans-(3,4dihydroxy-phenyl)-acryloyl]-methyl-amino}-propionic acid ethyl ester;
39) 3-(3,4-dihydroxy-phenyl)-(S)-2-{[3-trans-(3,4dihydroxy-phenyl)-acryloyl]-methyl-amino}-propionic acid propyl ester;
40) 3-(3,4-dihydroxy-phenyl)-(S)-2-{[3-trans-(3,4dihydroxy-phenyl)-acryloyl]-methyl-amino}-propionic acid isopropyl ester;
41) 3-(3,4-dihydroxy-phenyl)-(S)-2-{[3-trans-(3,4dihydroxy-phenyl)-acryloyl]-methyl-amino}-propionic acid tert-butyl ester;
42) N-[1-carbamoyl-2-(3,4-dihydroxy-phenyl)-ethyl](S)-3-trans-(3,4-dihydroxy-phenyl)-N-methyl-acrylamide;
43) 3-(3,4-dihydroxy-phenyl)-N-[2-trans-(3,4-dihydroxy-phenyl)-ethyl]-acrylamide;
44) 3-(3,4-dihydroxy-phenyl)-N-[2-(3,4-dihydroxy-phenyl)-ethyl]-N-methyl-acrylamide;
45) (R)-2-[trans-3-(3,4-dihydroxy-phenyl)-acryloylamino]-3-(4-hydroxy-phenyl)-propionic acid methyl ester;
46) (R)-2-[trans-3-(3,4-dihydroxy-phenyl)-acryloylamino]-3-(4-hydroxy-phenyl)-propionic acid;
47) (R)-2-[trans-3-(3,4-dihydroxy-phenyl)-acryloylamino]-3-(4-hydroxy-phenyl)-propionic acid methyl ester;
48) (S)-2-[trans-3-(3,4-dihydroxy-phenyl)-acryloylamino]-3-(4-hydroxy-phenyl)-propionic acid
49) (S)-2-[3-(3,4-dihydroxy-benzyl)-ureido]-3-(3,4-dihydroxy-phenyl)-propionic acid methyl ester;
50) 3-(3,4-dihydroxy-phenyl)-2-[2-(3,4-dihydroxy-phenyl)-acetylamino]-propionic acid methyl ester;
51) 2-(3,4-dihydroxy-benzoylamino)-3-(3,4-dihydroxy-phenyl)-propionic acid methyl ester;
52) 3-(3,4-dihydroxy-phenyl)-2-[3-(3,4-dihydroxy-phenyl)-propionylamino]-propionic acid methyl ester;
53) 3-(3,4-dihydroxy-phenyl)-2-[3-(3,4-dihydroxy-phenyl)-arylamino]-propionic acid methyl ester;
54) (R)-3-(3,4-dihydroxy-phenyl)-acrylic acid 2-(3,4-dihydroxy-phenyl)-1-methoxycarbonyl ethyl ester;
55) (R)-3-(3,4-dihydroxy-phenyl)-acrylic acid 2-(3,4-dihydroxy-phenyl)-1-propoxycarbonyl ethyl ester;
56) (R)-3-(3,4-dihydroxy-phenyl)-acrylic acid 2-(3,4-dihydroxy-phenyl)-1-tert-butoxycarbonyl ethyl ester;
57) (R)-3-(3,4-dihydroxy-phenyl)-acrylic acid 2-(3,4-dihydroxy-phenyl)-1-carbamoyl ethyl ester; and
58) (R)-3-(3,4-dihydroxy-phenyl)-acrylic acid 2-(3,4-dihydroxy-phenyl)-1-isopropylcarbamoyl ethyl ester.

4. A pharmaceutical composition for use in inhibiting activation of src homology region 2 domain of T lymphocyte cell kinase comprising the derivatives or their pharmaceutically acceptable salts of claim 1 as an effective ingredient.

5. A pharmaceutical composition for use in inhibiting immune responses comprising the derivatives or their pharmaceutically acceptable salts of claim 1 as an effective ingredient.

6. The pharmaceutical composition according to claim 5, wherein the composition is used for the treatment, prevention or diagnosis of rejection of transplanted organ or tissue, chronic rejection, or graft-versus-host diseases (GVHD).

7. The pharmaceutical composition according to claim 5, wherein the composition is used for the treatment, prevention or diagnosis of autoimmune diseases.

8. The pharmaceutical composition according to claim 7, wherein autoimmune diseases comprise lupus erythematosus, systemic erythematosus, rheumatoid arthritis, diabetes, myasthenia gravis, multiple sclerosis or psoriasis.

9. The pharmaceutical composition according to claim 5, further comprising one or more immunosuppressive drugs.

10. The pharmaceutical composition according to claim 9, wherein the immunosuppressive drugs is selected from the group consisting of cyclosporin A and its analogue, FK506 and its analogue, corticosteroid, azathioprine, mycophenolic acid, rapamycin, 15-deoxyspergualin, mizorubine, leflunomide, OKT3, IL-2 receptor antibodies, misoprostol, methotrexate, cyclophosphamide, anti-lymphocyte/thymocyte antisera, prednisone and methylprednisone.

11. A pharmaceutical composition for use in anti-inflammatory drugs comprising the derivatives or their pharmaceutically acceptable salts of formula 1 as an effective ingredient.

12. The pharmaceutical composition according to claim 11, further comprising one or more commonly anti-inflammatory drugs.

13. The pharmaceutical composition according to claim 12, the anti-inflammatory agent is selected from the nonsteroidal anti-inflammatory drugs consisting of aspirin, ibuprofen, naproxen, indomethacin, diclofenac, sulindac, piroxicam, etodolac, ketoprofen, meclofenamate, suprofen and tolmetin.

14. A pharmaceutical composition for use in treating arthritis comprising the derivatives or their pharmaceutically acceptable salts of formula 1 as an effective ingredient.

15. A method for preparing the derivatives of claim 1 including intramolecular amide bond by means of condensation of the carboxyl compound of formula 3 with amine compound of formula 2 in the presence of a coupling reagent and a base. 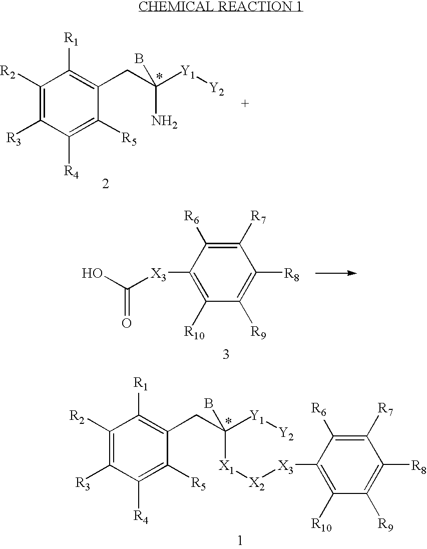
Wherein, R₁, R₂, R₃, R₄, R₅, R₆, R₇, R₈, R₉, R₁₀, X₁, X₂, X₃, Y₁, Y₂, B and * are the same as defined in claim 1.

16. The method according to claim 15, wherein the coupling reagent is selected from the group consisting of benzotriazole-1-yl-oxytripyrollidine phosphonium hexafluorophosphate and bromo-1-tripyrrolidine phosphonium hexafluorophosphate

17. The method according to claim 15, wherein the base is selected from the group consisting of p-dimethylaminopyridine, triethylamine and diisoethylamine.
